(19) United States
(12) Patent Application Publication
(10) Pub. No.: US 2020/0071699 A1
(43) Pub. Date: Mar. 5, 2020

(54) TREATMENT OF ALZHEIMERS DISEASE WITH MICRO RNA AND GHRELIN

(71) Applicants: Ghassan S. Kassab, La Jolla, CA (US); Lijuan Fu, San Diego, CA (US)

(72) Inventors: Ghassan S. Kassab, La Jolla, CA (US); Lijuan Fu, San Diego, CA (US)

(21) Appl. No.: 16/559,568

(22) Filed: Sep. 3, 2019

Related U.S. Application Data
(60) Provisional application No. 62/725,890, filed on Aug. 31, 2018.

(51) Int. Cl. C12N 15/113; A61K 31/7105; A61K 9/00; A61K 38/22; A61P 25/28; C12N 15/86; C12N 7/00

(52) U.S. Cl. CPC C12N 15/113; A61K 31/7105; A61K 9/0043; A61K 9/0019; A61K 38/22; C12N 2750/14171; C12N 15/86; C12N 7/00; C12N 2310/141; C12N 2750/14143; A61P 25/28

(57) ABSTRACT

Treatment of Alzheimer's disease with microRNA and ghrelin. In an embodiment of a product for treating Alzheimer's disease, the product includes recombinant adeno-associated virus (rAAV) vectors containing at least one microRNA (miRNA) sequence, wherein the at least one miRNA sequence is selected from the group consisting of miR-126, miR-145, miR-195, miR-21, and miR-29b.

### PRIORITY

The present patent application is related to, and claims the priority benefit of, U.S. Provisional Patent Application Ser. No. 62/725,890, filed on Aug. 31, 2018, the contents of which are hereby incorporated by reference in their entirety into this disclosure.

### BACKGROUND

Alzheimer's disease (AD), as one of the most important neurodegenerative disorders, is a world-wide problem that has no cure. It is reported that over 40% of US individuals above 85 years old have been diagnosed with AD. Alzheimer's disease (AD) affects 5.5 million Americans with the staggering societal burden and healthcare expenditures exceeding $236 billion.

The causes of most AD cases are still not fully understood except for the ˜5% of early-onset familial AD (FAD) cases that have been identified by aberrant alleles. Clinically, validated treatments for AD currently function by temporarily ameliorating symptoms of memory loss and improving behavioral disturbances. In the last decades, massive investment in AD drug development has been most commonly focused on neurochemical enhancers and the modulators of amyloid production pathways, including monoclonal antibodies against amyloid beta (Aβ) and the secretase inhibitors, which have experienced setbacks and failures.

Thus, it is urgent to investigate intervention strategies targeting novel modulators to develop innovative pharmaceutic therapies for AD to counter the increasing AD populations and healthcare costs. Several challenges, particularly the significant gaps of knowledge in the biological mechanisms of AD, however, impede the discovery of effective drugs for AD treatment. The causes of most AD cases are still not fully understood except for the ˜5% of early-onset familial AD (FAD) cases that have been identified by aberrant alleles.

There are several competing hypotheses to explain the cause of AD. The general consensus, namely “Amyloid (Aβ) Cascade Hypothesis”, suggests that the imbalance of Aβ metabolism promotes the aggregation of Aβ in the brain, initiating the neurodegeneration and cognitive impairment in AD. The proponents advocate the overproduction and processing of Aβ precursor protein (APP) as well as the failure elimination of Aβ from the brain lead to the accumulation of Aβ peptide which condenses and becomes insoluble fiber (fibril) to form senile plaque, resulting in denaturing of the neurons and developing the symptoms of this AD. A series of clinical trials based on amyloid reduction therapy failed to deliver the anticipated clinical improvement on mild-to-moderate patients with AD, raising legitimate concerns for the validity of this hypothesis.

The recent “oligomer hypothesis” suggests that the condensation process of soluble Aβ oligomer causes steady memory loss mediated by its synaptic injurious effect. Several different forms of Aβ, such as monomers, oligomers, and fibrils, exist in AD brains and are constantly dynamic. Several possible clearance systems that act together to drive extracellular soluble Aβ oligomers from the brain have been described in previous studies. These include enzymatic degradation, cellular uptake, blood-brain barrier (BBB) transportation, interstitial fluid (ISF, that surrounds neurons) bulk flow, and cerebrospinal fluid (CSF, that surrounds the brain) absorption by the circulatory and lymphatic drainage. The perivascular route exists in the spaces around the brain vasculature and is a path for delivering all the essential substances the cells require and allows the efflux of unwanted wastes, such as Aβ, through the ISF bulk flow. Soluble Aβ oligomers in the ISF flow are driven by arterial pulsation into the perivascular space located along the smooth muscle cells (SMCs) and capillary basement membrane and towards the subarachnoid space, and ultimately out of the brain. A study published in Nature showed that the extracellular 56-kDa soluble Aβ oligomer (Aβ *56) was the major culprit to disrupt the memory via Aβ *56-activated NMDAR-CaMKIIα-tau pathway. In addition, neurovascular network damage in AD has been suspected for a long time

A large body of data indicates that brain blood vessel deficit is a vital pathological trait of AD among the earliest clinical biomarkers. “Two-hit vascular hypothesis for AD” suggests signs of cerebrovascular pathology may be the initial steps of AD process. Dysfunctional cerebral vasculature may promote faulty Aβ clearance and precede the appearance of Aβ-initiated neuronal injury and cognitive impairment.

Given the long-time interval from the occurrence of pathological changes to AD manifestations, early interventions for AD is likely to succeed if therapy targets the initial cerebrovascular pathology in the disease process. Unfortunately, current clinical treatments for this disease act by temporarily ameliorating symptoms of memory loss and improving behavioral disturbances.

The “two-hit vascular hypothesis for AD” and “amyloid oligomer hypothesis” suggest that signs of cerebrovascular pathology could promote the imbalance of Aβ metabolism and the aggregation of Aβ in the brain, which may be the initial steps of AD pathogenesis and precede the appearance of Aβ-initiated neuronal injury and cognitive impairment.

In recent years, a large body of data proves that the damage of the cerebral vasculature is emerging as a key pathological trait of AD among the earliest clinical markers, notably late-onset sporadic AD (SAD) that accounts for more than 99% of AD cases. Therefore, novel strategies for potential prevention and/or therapeutics for AD (especially in the early phase of AD) concentrated on the subsequent elimination of Aβ through the cerebral vasculature raises new hope for this devastating disease. The multifaceted pathogenesis of AD implicates a complex interaction among numerous insults and cell types. The complexity of the cerebrovascular network requires coordinated genetic programs that are partly controlled by transcriptional activity. The endogenous non-coding RNAs, such as microRNAs (miRNAs, ˜22nt) that modulate gene expression in series of the biological program, are a promising approach to treat many complex multi-factors diseases and modify multiple-actions through regulating multiple-molecular cascades. Currently, miRNAs have been utilized to impact both cardiovascular diseases and AD. MiR-126 is most highly enriched in endothelial cells (ECs), involved in the angiogenesis, vascular activation, inflammation and vascular tone, as well as control of transport barrier function.

### BRIEF SUMMARY

The expression of miR-126 orchestrates accurate tuning of gene expression that contributes to ECs homeostasis and BBB integrity, further influencing on the extracellular Aβ clearance, synaptic functions, and cognitive behavior. The studies referenced herein confirmed that a change in the levels of this miRNA affects the clearance of Aβ and rescues the dysfunction of the cerebral vasculature, providing a therapeutic rationale. Thus, one embodiment of the current invention is targeting miR-126 in AD mice brains through noninvasive nose-to-brain delivery route. EC-specific miR-126 provides a novel, readily accessible preventative and/or therapeutic method for AD treatment to substantially reduce the associated healthcare costs.

The embodiment of novel pharmacologic intervention aimed at altering the levels of cerebrovascular miRNAs will impact AD with heterogenic or epigenetic origin.

Our observations suggest the abnormal structure and function of capillaries in AD mice brains were associated with abnormal levels of capillary miRNAs. Additionally, we made a novel finding that the upregulation of miR-126 specifically promoted the elimination of Aβ*56 that plays a crucial role in the activation of NMDAR-CaMKIIα-tau neuronal signaling and synaptic function in AD brains

Recombinant AAV with the character of low immunogenicity, non-toxicity, high transfection efficiency and long-term stable expression (at least one year), etc., is a vector used to mediate the delivery of miRNA mimic or inhibitor into cells.

The studies referenced herein validate the effect of treatment targeting miR-126 in AD mice brains. To accomplish this objective, a specific aim is to verify the safety (primarily) and efficacy (secondarily) of adeno-associated virus (AAV) containing miR-126 mimic through noninvasively intranasal delivery in early-onset and late-onset transgenic AD mice models. Our study on EC-specific miR-126 provides a novel, accessible preventative and/or therapeutic method for AD treatment and associated healthcare costs.

In an exemplary embodiment of a product for treating Alzheimer's disease of the present disclosure, the product comprises recombinant adeno-associated virus (rAAV) vectors containing at least one microRNA (miRNA) sequence, wherein the at least one miRNA sequence is selected from the group consisting of miR-126, miR-145, miR-195, miR-21, and miR-29b.

In an exemplary embodiment of a product for treating Alzheimer's disease of the present disclosure, the at least one miRNA sequence is miR-126.

In an exemplary embodiment of a product for treating Alzheimer's disease of the present disclosure, the at least one miRNA sequence further comprises at least one additional miRNA sequence.

In an exemplary embodiment of a product for treating Alzheimer's disease of the present disclosure, the product is configured for intranasal administration

In an exemplary embodiment of a product for treating Alzheimer's disease of the present disclosure, the at least one miRNA sequence is miR-145.

In an exemplary embodiment of a product for treating Alzheimer's disease of the present disclosure, the at least one miRNA sequence further comprises at least one additional miRNA sequence.

In an exemplary embodiment of a product for treating Alzheimer's disease of the present disclosure, wherein the miRNA sequence is vessel-specific.

In an exemplary embodiment of a method of the present disclosure, the method comprises the step of administering the product to an individual having Alzheimer's disease to treat the Alzheimer's disease.

In an exemplary embodiment of a method of the present disclosure, the step of administering is performed using intranasal administration.

In an exemplary embodiment of a method of treating Alzheimer's disease of the present disclosure, the method comprises the step of administering at least one recombinant adeno-associated virus (rAAV) vector to an individual having Alzheimer's disease, wherein the rAAV vector comprises at least one microRNA (miRNA) sequence, wherein the at least one miRNA sequence is selected from the group consisting of miR-126, miR-145, miR-195, miR-21, and miR-29b.

In an exemplary embodiment of a method of treating Alzheimer's disease of the present disclosure, the step of administering is performed using intranasal administration.

In an exemplary embodiment of a method of treating Alzheimer's disease of the present disclosure, the method further comprises the step of administering ghrelin to the individual prior to the step of administering at least one rAAV vector.

In an exemplary embodiment of a method of treating Alzheimer's disease of the present disclosure, the method further comprises the step of administering ghrelin to the individual after the step of administering the at least one rAAV vector.

In an exemplary embodiment of a method of treating Alzheimer's disease of the present disclosure, the step of administering further comprises administering ghrelin.

In an exemplary embodiment of a method of treating Alzheimer's disease of the present disclosure, the method further comprises the step of administering ghrelin subcutaneously.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1A shows images of the exclusive perivascular accumulation of Aβ in 3×Tg mice brains aged 6 months, with boxes A-H being A11 positive (red in the color photographs from the original data) blood vessels labeled with Lectin (green in the color photographs from the original data set) in the cerebral cortex and hippocampus of WT (boxes A-D) and 3×Tg (boxes E-H), with mice aged 6 months (boxes A, B, E, and F), 9 months (boxes C and G) and 12 months (boxes D and H), and with boxes I and J showing Aβ plague stained with 6E10 (box I) antibody (red in the color photographs from the original data) and cerebrovascular aggregated Aβ (CAA) marked with 4G8 (box J) antibody (red in the color photographs from the original data) in 20 month old AD mice brain, whereby A11 antibody was used to stain the high molecular weight Aβ oligomers, with blue (in the color photographs from the original data) Hoechst 33342 stained nuclear, and with the scale bar being 20 μm, according to exemplary embodiments of the present disclosure;

FIG. 1B shows graphical data, with section K showing the capillary density of WT and AD (3×Tg-AD) mice, and with section L showing the quantitative analysis for the number of A11⁺ vessels in WT and AD brains, with error bars=SEM, *P<0.05, ***P<0.001, Student's t test, and n=5 animals/group, according to exemplary embodiments of the present disclosure;

FIG. 2A shows images of increased CD3ε⁺ vessels adjacent to cerebral vasculature in young AD mice brains, with boxes A-F being the representative images of CD3ε (red in the color photographs from the original data) expressing blood vessels in the cerebral cortex and hippocampus of WT (boxes A, C, and E) and 3×Tg (boxes B, D, and F), of mice aged 6 months (boxes A and B), 9 months (boxes C and D), and 12 months (boxes E and F), with green (in the color photographs from the original data) being Lectin labeled blood vessels, with blue (in the color photographs from the original data) Hoechst 33342 stained nuclear, and with the scale bar being 20 μm, according to exemplary embodiments of the present disclosure;

FIG. 2B shows graphical data, with section G showing the quantitative comparison of the number of CD3ε⁺ vessels between 3×Tg and WT mice, with error bars=SEM, ***P<0.001, Student's t test, and n=5 animals/group, according to exemplary embodiments of the present disclosure;

FIG. 3A shows images of increment of CD68 ⁺ blood vessels observed in young AD mice brains, with boxes A-F showing CD68⁺ red (in the color photographs from the original data) blood vessels in the cerebral cortex and hippocampus of WT (boxes A, C, and E) and 3×Tg (boxes B, D, and F), with mice aged 6 months (boxes A and B), 9 months (boxes C and D), and 12 months (boxes E and F). with green (in the color photographs from the original data) being Lectin labeled blood vessels; with blue (in the color photographs from the original data) Hoechst 33342 stained nuclear, and with the scale bar being 20 μm, according to exemplary embodiments of the present disclosure;

FIG. 3B shows graphical data, with section G showing the measurement for the number of CD68 ⁺ vessels in 3×Tg and WT mice brain, with the error bars=SEM, ***P<0.001, Student's t test, and n=5 animals/group, according to exemplary embodiments of the present disclosure;

FIG. 4 shows images of various expression patterns of Aβ using different antibodies in AD mice brain, with boxes A-C showing cerebral cortex and hippocampus sections from 3×Tg-AD mice that were stained by 6E10 (green in the color photographs from the original data) at the ages of 6 months (box A), 9 months (box B), and 12 months (box C), with the rectangle showing the magnified area of the hippocampus CA1 regions (boxes D-F), with the hippocampus areas stained by anti-Aβ fibril antibodies red (in the color photographs from the original data) at the ages of 6 months (box D), 9 months (box E), and 12 months (box F), with boxes G-I showing the high molecular weight Aβ oligomers in CA1 regions of hippocampus areas that were stained with A11 antibody at the ages of 6 months (box G), 9 months (box H), and 12 months (box I), with green (in the color photographs from the original data) being Lectin labeled blood vessels; with blue (in the color photographs from the original data) Hoechst 33342 stained nuclear, and with the scale bar being 50 μm in boxes A-C and G-1 and being 10 μm in boxes D-F, according to exemplary embodiments of the present disclosure;

FIG. 5 shows the percentage of pericytes coverage in the wild type and AD mice, with box A showing a phase contrast image showed the morphology and purification of isolated microvessels, with box B showing the endothelial cells and pericytes of isolated capillaries were labeled with Lectin (green in the color photographs from the original data) and PDGFRβ (red in the color photographs from the original data) antibody respectively in AD mice, with the scale bar being 10 μm in box A and 5 μm in box B, and with arrows indicating pericytes, and with subsection C showing a quantitative comparison of pericytes coverage of capillaries profiles between WT and AD (3×Tg-AD) mice at the ages of 6 through 12 months (n=6 animals/group), with error bars=SEM, *P<0.05, **P<0.01, ***P<0.001, and Student's t test, according to exemplary embodiments of the present disclosure;

FIG. 6 shows comparative expressions of 5 miRNAs in isolated capillaries, with subsections A-C showing the quantitative comparison of qPCR results of 5 miRNAs levels in isolated capillaries of WT and AD (3×Tg-AD) mice at 6 months (subsection A), 9 months (subsection B), 12 months (subsection C), with n=6 animals/group, and with subsection D showing the abundance of 5 miRNAs in isolated capillaries showed as a representative result of threshold cycle numbers from a WT animal of 9 months, with error bars=SEM, *P<0.05, **P<0.01, and Student's t test, according to exemplary embodiments of the present disclosure;

FIG. 7A shows the effect of Ghrelin on pericytes coverage, miRNAs and Aβ levels in 3×Tg mice aged 9 months, with boxes A and B showing perictyes coverage of capillaries isolated from the hippocampus and cerebral cortex of 3×Tg mice injected by vehicle (box A, saline) or ghrelin (box B), with green (in the color photographs from the original data) being Lectin labeled ECs, with red (in the color photographs from the original data) being PDGFRβ antibody stained perictyes, with the scale bar 5 μm, and with subsection C showing quantified pericytes coverage in ghrelin and saline treated groups, according to exemplary embodiments of the present disclosure;

FIG. 7B shows graphical and blot information, with subsection D showing expression of 5 selective microvessels miRNAs in ghrelin (3×Tg-G) and saline (3×Tg-S) injected AD mice, with subsection E showing Aβ oligomers measured by ELISA, and with subsection F showing representative images for Aβ levels in the vessels-depleted hippocampus and cerebral cortex of 3×Tg mice treated with ghrelin (G) or saline (S), with sAPP, soluble APP, with GAPDH as loading control, with error bars=SEM, *P<0.05, ***P<0.001, Student's t test; and n=6-8 animals/group, according to exemplary embodiments of the present disclosure;

FIG. 8A shows images of Ghrelin treatment that diminished the expression of RAGE, with boxes A-F being representative of images of LRP1 (boxes A and B), RAGE (boxes C and D) and Mdr1 (boxes E and F) staining in isolated capillaries from saline (boxes A, C, and E) or ghrelin (boxes B, D, and F) animals, according to exemplary embodiments of the present disclosure;

FIG. 8B shows data of the percent area of lectin-positive microvessels occupied by LRP1, RAGE, and Mdr1, where immunofluorescent signals were quantified in saline and ghrelin treated 3×Tg mice, with nor bars=SEM, *P<0.05, Student's t test, and n=6-8 animals/group, according to exemplary embodiments of the present disclosure;

FIG. 9 shows data in connection with 11 total cerebral capillaries miRNAs that were screened, with subsection A showing the quantitative comparison of qPCR results of 11 miRNAs levels in isolated microvessels of WT and AD mice (n=3-5 animals/group), whereby only 3 miRNAs, i.e. miR-126, 145, and 195 were significantly upregulated in 3×Tg mice aged 5 months old compared with WT mice at the same age, noting that no expression for miR-199 and 208 was found in either WT or 3×Tg mice brain capillaries, and with subsection B showing the abundance of 11 miRNAs in isolated capillaries showed as a representative result of threshold cycle numbers from a WT animal of 6 months, with error bars=SEM, *P<0.05, **P<0.01, and Student's t test, according to exemplary embodiments of the present disclosure;

FIG. 10 shows data in connection with cerebrovascular reactivity in aged (20 months) 3×Tg-AD mice, whereby middle cerebral arteries were studied using pressure myography techniques (60 mmHg), with subsection A showing that contractions to 60 mM KCl were similar in WT and 3×Tg-AD mice aged 20 months, indicating that vessels smooth muscle cells function was intact, and with subsection B showing that relaxation to acetylcholine (ACh) was impaired in aged 3×Tg-AD mice, indicating endothelial dysfunction, with error bars=SEM and n=2-3 animals/group, according to exemplary embodiments of the present disclosure;

FIG. 11 depicts information relating to intranasal drug administration, with subsection A showing a diagram of nose-to-brain drug delivery, box B showing intranasal AAV-miR-126 administration, and box C showing the representative image for the infection efficiency of AAV vectors after 3 days administration, with the colocalization of eGFP expressed AAV vectors (green in the color photographs from the original data) with Lectin labeled capillaries (red in the color photographs from the original data), with a Hoechst 33342 stained nucleus, according to exemplary embodiments of the present disclosure;

FIG. 12 shows a comparison of 5 miRNAs expressions in isolated capillaries and Aβ deposited capillaries of WT (B6 129) and AD (3×Tg-AD) female mice, with subsections A and B showing a quantitative comparison of qPCR results of 5 miRNAs levels in isolated capillaries of the hippocampus and cortex (n=6) at 6 months (subsection A) and 9 months (subsection B), and with subsection C showing the quantitation of high molecular weight Aβ oligomer-specific antibody (A11) stained capillaries (n=6), with error bars=SEM, *p<0.05, **p<0.01, and Student's t-test, according to exemplary embodiments of the present disclosure;

FIG. 13A shows levels of miRNAs, BBB permeability and BBB breakdown in 6mo GFAP-ApoE4 and WT (C57BL/6J) mice, with subsection A showing the quantitative qPCR results of selective miRNAs levels in isolated capillaries of the hippocampus and cortex, and with subsection B showing BBB permeability determined by Evans Blue spectrophotometry, with error bars=SEM, *p<0.05, Student's t-test, and n=6, according to exemplary embodiments of the present disclosure;

FIG. 13B shows photographs of extravascular thrombin deposits (red in boxes C and D, and green in boxes E and F, colors from the original data) in mice brains, with lectin-positive capillaries in boxes C and D (green in the color photographs from the original data), and Neu N marked neuronal bodies in boxes E and F (red in the color photographs from the original data), with Hoechst 33342 labeled nucleus (blue in the color photographs from the original data), with scale bar being 5 μm, according to exemplary embodiments of the present disclosure;

FIGS. 14A and 14B show the effect of Ghrelin and LNA inhibitors on cerebrovascular miRNAs and Aβ levels in 3×Tg mice aged 9 months (subsections A and C and 6 months (subsections B and D), with subsection A showing the expression of 5 selective microvessels miRNAs between ghrelin (3×Tg-G) and saline (3×Tg-S) injected AD mice, with subsection B showing qPCR results of miR-126,145 and 21 levels in isolated capillaries of inhibitors treated AD mice and control group (scramble), with error bars=SEM; *p<0.05, ***p<0.001, Student's t-test, and n=6, and with subsection C showing the representative images for APP and Aβ oligomers levels in the microvessel-free hippocampus and cerebral cortex of 3×Tg mice treated with ghrelin (G) or saline (S) using 6E10 and A11 antibodies, and with subsection D showing immunoreactivities of high molecular weight oligomers of Aβ in the microvessel-free hippocampus and cerebral cortex of inhibitors (In-1 and In-2) and scramble (NC) treated mice by using oligomer-specific antibody (A11), with GAPDH as loading control, and sAPP (soluble APP), according to exemplary embodiments of the present disclosure;

FIGS. 15A, 15B, and 15C show the effect of AAV vectors on Aβ 56* oligomers levels in 3×Tg (subsection A, boxes/rows C and D, and subsection E) and apoE4 (subsections B and F) AD mice, with subsections A and B showing the level of Aβ 56* (indicated as the arrow) in the brain and liver of 3×Tg mice (subsection A) and the brain of apoE4 mice (subsection B) between animals treated with AAV-miR-126 and AAV blank vectors, with boxes/rows C and D showing the phosphorylated signal of CAMKII (green in the color photographs from the original data) in the hippocampus (box/row C) and cerebral cortex (box/row D) of AAV-miR-126 and control 3×Tg mice groups, with PDS-95 positive synapses in red (in the color photographs from the original data), with Hoechst 33342 labeled nucleus (blue in the color photographs from the original data), scale bar: 10 μm, and with subsections E and F showing the decreased signal of p-CAMKII-α in AAV-miR-126 infected 3×Tg (subsection E) and apoE4 (subsection F) mice, with GAPDH as the loading control, n=1-4, control as AAV blank vectors, and 126 being AAV-miR-126, according to exemplary embodiments of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The utility of cerebral ECs-specific miR-126 as a therapeutic target in AD treatment is novel. The proposed work will, for the first time, validate the effect of this miRNAs in the cerebral vasculature dysfunction of AD animal model. Furthermore, miRNA modulators in the blood can reach their targets, namely ECs, smooth muscle cells and pericytes, much more easily than other potential drugs targeting neuronal or glial cells. The innovative and clinically significant feature of this proposal resides in both the efficient and stable transduction for ECs-specific miR-126 with little immunogenicity or toxicity and the noninvasive administration method. Specifically, the intranasal route prevents the injury caused by brain surgery and the systemic absorption. Validation of this novel approach as referenced herein provides a therapeutic alternative for AD treatments before the brain starts to deteriorate.

AAV is a small non-pathogenic virus single-stranded DNA parvovirus containing 4.7 kilobases genome in length. Recombinant AAVs (rAAV) have become effective tools for use as gene therapy vectors for several reasons. First, rAAV causes no known pathogenic disease in infected humans. Second, rAAVs are capable of transducing a variety of tissues and cell types including brain, blood vessels, neurons and ECs. Third, rAAVs are able to maintain stable expression of transgenes for periods greater than 1.5 years in various animal models including rodents and large animals. Fourth, the low frequency of viral integration reduces the likelihood of insertional mutagenesis. AAV serotype 2 (AAV2) had been used to date in 75 clinical trials worldwide, including 14 trials to treat neurological disease including Parkinson's disease, AD, amyotrophic lateral sclerosis, and epilepsy.

Most treatments for neurological diseases are ineffective due to the inability to traverse the BBB. As an alternate, nasal instillation can be used as an efficient and clinically amenable treatment to delay the onset of CNS disorders. The nasal cavity is the only site in the mammalian body where CNS is in direct contact with the surrounding environment. Such strategy will avoid brain surgery and allow gene therapy to affect a large portion of the brain, sparking interest as a potential therapeutic approach for AD. It has been applied successfully in many studies to transduce CNS, increasingly getting more attention for delivery of wide variety of drug molecules. The formulations ranging from small molecules to large molecules such as nucleotides, peptides, and proteins gain direct access to the brain. This route takes full advantage of preventing the enzymatic degradation and enhancing the pharmacological effects without systemic absorption as well as avoiding the toxicity to the major peripheral organs. An intranasally delivered peptide drug has been demonstrated to ameliorate cognitive decline in Alzheimer transgenic mice.

Intranasal route of AAV transportation allows to directly deliver therapeutic molecules rapidly to the brain via olfactory and trigeminal pathways without systemic absorption, avoiding the side effects and enhancing the efficacy of neurotherapeutics. Small and large molecules delivered through the nose, can access the brain in therapeutic concentrations, serving as an effective delivery method for central nervous system (CNS) drugs that bypass the BBB. This innovative treatment targeting miR-126 carried by AAV vectors via nasal-to-brain delivery will provoke minimal inflammatory response and produce stable expression by a lower dosage and virus titers with strengthening drug efficacy. Thus, a noninvasive strategy for intranasal AAV1 (serotype 1)-miR-126 instillation imparts ease of administration, rapid onset of action, and avoids first-pass metabolism as well as the adverse effect of brain surgery. Direct transport of ECs-specific miRNA drugs along the olfactory and trigeminal nerves can be potentially used to control Aβ levels in the brain to treat AD manifestations, as well as be considered as an important and promising therapeutic approach. Therefore, the success of this Phase I project aimed to upregulate vascular miRNA in AD mice brains will stimulate translational investigations on the large animal for the treatment of AD and facilitate the development of an effective and safe pharmaceutical intervention for clinical studies of this devastating disease.

### I. First Study

How cerebrovascular miRNAs regulate the expression of intracellular genes of the vessel wall, which in turn affect Aβ oligomer aggregation in AD brains remains unknown. To address this question, we have screened 11 capillary miRNAs closely related with both cardiovascular diseases and AD. The 5 most abundant and significantly changed miRNAs were selected to analyze the relationship between the functional activation of the cerebral vasculature and their expression patterns in different AD phases of 3×Tg mice. Ghrelin, known as the “hunger hormone”, is a neuropeptide generated from ghrelinergic cells of the gastrointestinal tract. It has numerous functions, including appetite stimulation, increase in food intake and fat storage, as well as regulation of energy homeostasis. Furthermore, since ghrelin is thought to stimulate angiogenesis in ischemic muscles by inducing miRNAs upregulation, ghrelin was administered via subcutaneous injection to induce upregulation of miRNAs at the stage of lower vascular activities in AD brains to verify the relationship between selective vascular miRNAs and Aβ clearance. The findings of this work provide a more integrative understanding of the cellular and molecular progression in the pathology of AD which may enhance the development of cerebrovascular miRNA-targeting strategies aimed at ameliorating the dysfunction of brain blood vessels in AD brain.

### A. Materials and Methods

### Animals:

Triple transgenic mice, 3×Tg-AD, containing three mutations (PEN1 M146, APP Swedish and MAPT P301L), are widely used as an animal model of FAD. Age and gender-matched B6129SF2/J strain were used as the wild type (WT) control. Mice were obtained from the Jackson Laboratory and bred in our research institute's animal facility. Mice were housed in plastic cages on a 12 hr/12 hr light/dark cycle with ad libitum access to water and standard rodent diet. Animal usage was approved by California Medical Innovations Institute (CalMI2) Institutional Animal Care and Use Committee (IACUC). The genotyping was conducted in CalMI2 animal facilities at the age of 21 days by tail DNA extraction according to our previous protocol and the online information supplied by the vendor. Ten mice per strain (3×Tg-AD, B6129SF2/J) at the ages of 6, 9, and 12 months were used in this study. In the ghrelin administration study, 3×Tg-AD mice aged 9 months received s.c. injections of either n-octanoylated ghrelin (AnaSpec, 600m/kg per day, n=6-8) or saline every 2 days for 2 weeks.

### Capillaries Isolation:

Capillaries were isolated as previously described. Mouse brains were carefully isolated and the meninges were removed in ice-cold HBSS containing 1% BSA. The cortex and hippocampus were macroscopically dissected and all visible white matter was discarded. Tissues were then minced and homogenized in HBSS containing 1% BSA with a glass-douce homogenizer on ice. Dextran (70 kDa, Sigma) was subsequently added to yield a final concentration of 16% and the samples were thoroughly mixed, followed by centrifugation at 6,000 g for 15 min. The microvessel-depleted brain remaining on top of the Dextran gradient was collected for Aβ identification, and the capillary pellets located at the bottom of the tubes were harvested. Due to the small yields of capillaries per mouse, the capillary pellets from two animals were pooled and sequentially filtered through a 100 μm and 6 μm cell strainer (pluriSelect). The capillaries remaining on top of the 6 μm cell strainer were collected in HBSS buffer and either lysed to collect total RNA for real-time PCR or smeared on glass slides for fluorescent staining analysis.

### Microvessels Immunofluorescent Detection:

The isolated microvessel fragments were smeared onto Superfrost Plus pre-cleaned glass microscope slides and fixed using ICC Fixation Buffer (BD Pharmingen) for 15 min at room temperature (RT). The microvessels were then rinsed with PBS and blocked in PBS containing 0.3% Triton X-100 and 5% donkey serum (Jackson ImmunoResearch) for 1 hr at RT followed by incubation with the primary antibodies [mouse anti-PDGFRβ for staining pericytes, R&D systems; mouse anti-LRP1 (low-density lipoprotein receptor-related protein 1), RAGE (receptor for advanced glycosylation end products) and Mdr1 (multidrug resistance protein 1, also known as ABCB1) for detecting the transporters of Aβ on the BBB, Santa Cruz] overnight at 4° C. The slides were washed and incubated with the secondary antibodies (Alexa Fluor 546 conjugated donkey anti-mouse secondary antibody, Invitrogen) diluted in 1% donkey serum containing DyLight 488 Labeled Lycopersicon Esculentum Lectin (1:200, Vector Laboratories) for 1 hr at RT. For coverage analysis, the percentage of PDGFRβ-positive pericyte area covering lectin-positive capillary area was quantified using Image J Area analysis as described previously. For the expression analysis of Aβ transporters, the area of LRP1, RAGE and Mdr1-occupied endothelium were measured as an area percentage normalized by the total area of lectin-positive capillaries using Image J Area measurement tool. A total of 15-30 images were collected from each slide, and 6 mice per group were used for statistical analysis. Analysis of images was conducted blindly.

### Tissue Immunofluorescent Staining:

Mice were anesthetized with 1-2% isoflurane by inhalation. Intracardiac perfusion with 100 mM PBS (pH=7.4) containing 5 U/ml heparin was performed and followed by 4% fresh paraformaldehyde (PFA) in 100 mM PBS. The brains were dissected and maintained in 4% PFA at 4° C. until sectioning. Perfused brains were embedded into Richard-Allan Neg 50 Frozen Section Medium (Thermo Scientific) in liquid nitrogen. Embedded frozen brain tissue was cryo-sectioned at a thickness of 14 μm. For staining Aβ with 6E10 and 4G8 antibodies, sections were pretreated with formic acid solution (70%) at RT for 15 min to perform antigen retrieval. Then, sections were blocked with 5% donkey serum for 60 min and incubated with primary antibodies (6E10 and 4G8 for recognizing all forms of amyloid, Biolenged; A11 for detecting soluble Aβ oligomers, Rockland; anti-Aβ fibril, Abcam; anti-CD68 and CD3ε for mainly labeling the macrophages and T cells respectively, Santa Cruz) diluted in 1% donkey serum overnight at 4° C. Given the A11 antibody was produced from whole rabbit serum prepared by repeated immunizations with a synthetic molecular mimic of soluble oligomers according to manufacturer's instructions, it can specifically recognize all types of amyloid oligomers, but not detect native proteins, amyloidogenic monomers, or mature amyloid fibrils. Washed slides were incubated in secondary antibodies (Alexa Fluor 546 conjugated donkey anti-rabbit and anti-mouse secondary antibody, Alexa Fluor 488 conjugated donkey anti-mouse secondary antibody, Invitrogen) with DyLight 488 Labeled Lycopersicon Esculentum Lectin (1:200, Vector Laboratories) and Hoechst 33342 stain (1:5000, Invitrogen) 1 hr. at RT. Slides were washed, and coverslips were mounted by Shandon Immu-Mount (Thermo Scientific). Fluorescence was visualized and photographed by Nikon ECLIPSE TE300 with ISCapture software and Nikon ECLIPSE Ts2R with NIS Elements software. To analyze Aβ aggregated-vessels and vascular activation, the number of A11⁺, CD3ε⁺ and CD68⁺ vessels of Lectin-positive endothelium were counted and expressed as the average number of A11⁺, CD3ε⁺ and CD68⁺ vessels in Lectin-labeled endothelium. The capillary density was analyzed by counting the number of capillary branches. Five animals per group and 6-8 randomly selected fields from the cortex and hippocampus in 6 nonadjacent sections of each animal were used for statistical analysis.

### Reverse Transcription and Quantitative Real-Time PCR Analysis:

Total RNA was purified with Trizol reagent (Invitrogen), according to the manufacturer's instructions. To quantify miR-126-3p (MIMAT0000138), miR-145-5p (MIMAT0000157), miR-195-5p (MIMAT0000225), miR-21-5p (MIMAT0000530) and miR-29b-3p (MIMAT0000127), reverse transcription and quantitative PCR (qPCR) were performed using the TaqMan@ microRNA assay kit (Applied Biosystems) as previously described. Briefly, reverse transcription was performed in a 15 μl reaction mix containing 10 ng of total RNA, 3 μl of miRNA primer mix, 1 mM dNTP, 50 U reverse transcriptase, and 3.8 u. RNase inhibitor. Reactions were incubated at 16° C. for 30 min, and 42° C. for 30 min followed by 85° C. for 5 min. The PCR was performed in a 10 μl reaction volume containing 0.5 μl of miRNA primer and TaqMan probe mix, 0.67 μl of RT product (diluted fivefold), and 5 μl of TaqMan Universal PCR Master Mix. The cycling conditions were as follows: 10 min at 95° C. followed by 40 cycles of 15 s at 95° C. and 1 min at 60° C. U6 small RNA was used as an internal control following the manufacturer's recommendation. For all samples, reverse transcription and qPCR were performed three times and qPCR was performed in triplicate. Relative gene expression levels between wild type and 3×Tg-AD mice were determined using the comparative Ct (2^{-ΔΔCt}) method after normalizing to U6.

### Immunoblotting Analysis:

The method has been described previously. Briefly, microvessel-depleted cortex and hippocampus tissues (75 μg per lane) were homogenized in lysis and extraction buffer containing 50 mM β-glycerophosphate, 0.1 mM Na₃VO₄, 2 mM MgCl₂, 1 mM EGTA, 1 mM DTT, 0.02 mM pepstatin, 0.02 mM leupeptin, and 1 mM PMSF, as well as 0.5% Triton X-100 and 0.1 U/ml aprotinin. After centrifugation at 12,000 rpm for 20 min, protein content was determined by Bradford assay. Protein samples were separated by 4-12% Bis-Tris gels (Life Technologies) and then transferred to nitrocellulose membranes (Bio-Rad), which were blocked 1 hr. at RT with 5% bovine serum albumin Tris-buffered saline-Tween (0.5 M NaCl, 20 mM Tris-HCl, 0.1% (v/v) Tween 20, pH 7.6). Membranes were incubated overnight at 4° C. in buffer containing primary antibody (1:1000 for A11, Rockland, and 1:2000 for GAPDH, Santa Cruz) followed by horseradish peroxidase-conjugated secondary antibody. The A11 antibody was used to detect the high molecular weight Aβ oligomer specifically. GAPDH (a loading control) and A11 immunoreactivity were visualized with ECL Prime (Amersham) according to the manufacturer's instructions. The experiments were repeated at least three times.

### Aβ Oligomer Enzyme-Linked Immunosorbent Assay:

Human Aβ oligomers were analyzed in the microvessel-depleted cortex and hippocampal supernatant by enzyme-linked immunosorbent assay (ELISA; IBL International, Germany) according to manufacturer's instructions. The ELISA uses mouse monoclonal anti-human Aβ (N) (82E1) antibodies that recognize the N-terminus of human Aβ specifically, with 2 or more epitopes.

### Statistical Analysis:

All images were prepared using Adobe Photoshop CS5. Statistical analysis was performed using SPSS 21.0. Results were expressed as mean±SEM. The difference between two data sets was determined using Student's t-test, with P<0.05 indicating statistical significance.

### Cerebrovascular Reactivity:

Middle cerebral arteries and basilar artery of the mice were dissected, excised, and transferred into the myograph bath chamber filled with physiological salt solution (PSS; in mmol/L: 142 NaCl, 4.7 KCl, 2.7 sodium HEPES, 3 HEPES acid, 1.17 MgSO4, 2.79 CaCl2, and 5.5 glucose) and cannulated at two ends of tubes containing PSS. The vessel were stretched to in vivo length and equilibrated for 40 min with intravascular pressure set at 10 mmHg while the chamber temperature was gradually increased to 37° C. The vessel segments were exposed to cyclic transmural pressure from 100 to 0 mmHg. The diameter of the artery were calculated according to its internal circumference. Phenylephrine and acetylcholine dose-response contraction and dilation experiments were performed on each vessel at the aged (20 mo) AD and WT mice. The overall contractility of the vessel segments was tested with 60 mM KCl.

### B. Results

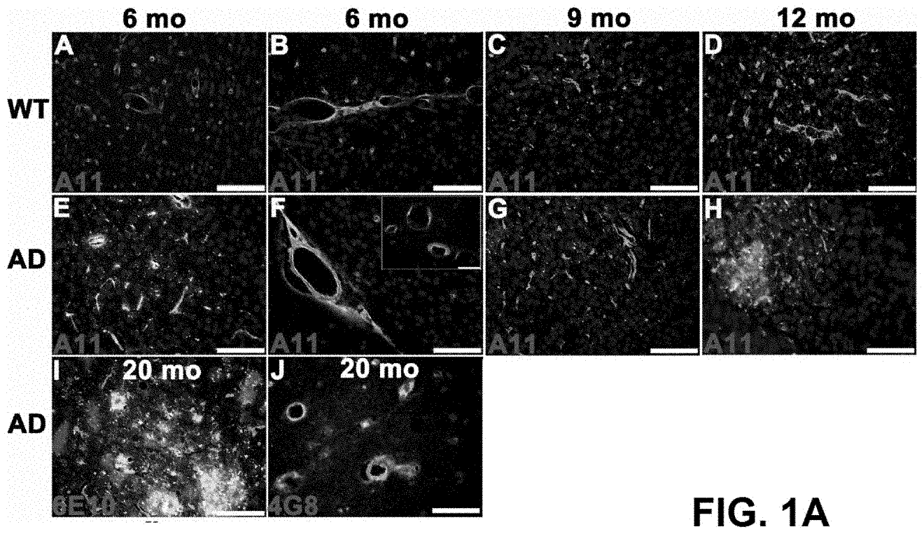

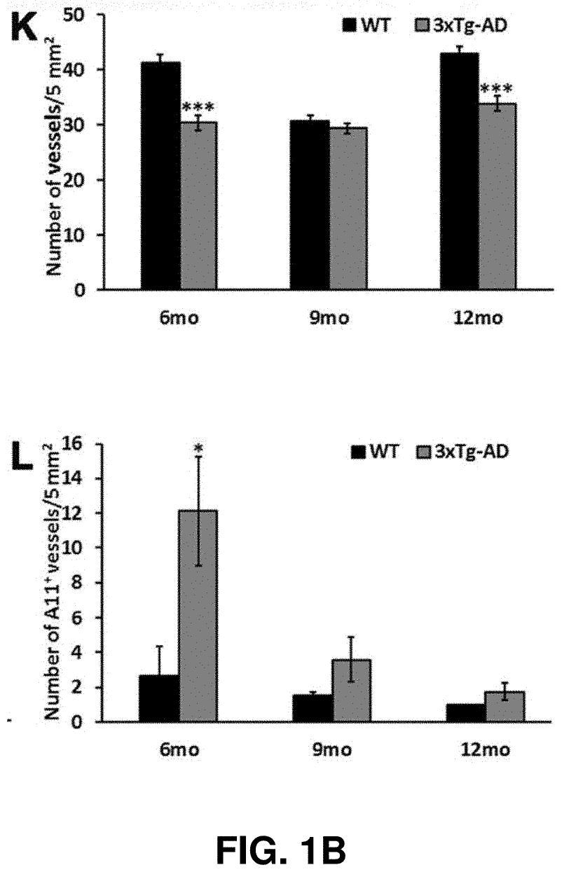

Aβ oligomer-laden cerebral blood vessels were present in young 3×Tg mice. The number of vessels per 5 mm² in the cerebral cortex and hippocampus were evaluated as the density of capillaries (diameter ≤10 μm, labeled by Lectin), which were significantly reduced (P<0.001) in AD mice brains aged at either 6 months or 12 months compared to that of WT mice at the same ages (shown in boxes E and H of FIG. 1A and quantified as shown in subsection K of FIG. 1B). Interestingly, the number of blood vessels stained with A11 antibody in younger AD mice (6 months) was much higher (P<0.05) than that of WT mice at the same age (subsection L of FIG. 1B). The significant increase in Aβ oligomers was exclusively observed in the perivascular space of both larger vessels (diameter >50 μm) and capillaries (box F of FIG. 1A) at the early stage of AD mice (6 months, boxes E and F of FIG. 1A), rather than the middle (9 months, box D of FIG. 1A) or late (12 months, box H of FIG. 1A) phases. The perivascular Aβ oligomer burden is reminiscent of cerebral amyloid angiopathy (CAA). Meanwhile, Aβ plaques appeared in both brain parenchyma (box I of FIG. 1A) and blood vessel walls (CAA in 20 months, box J of FIG. 1A) at the advanced stage of AD.

Activated Endothelium with Positive Immune Cells Around Cerebral Blood Vessels in Younger 3×Tg Mice.

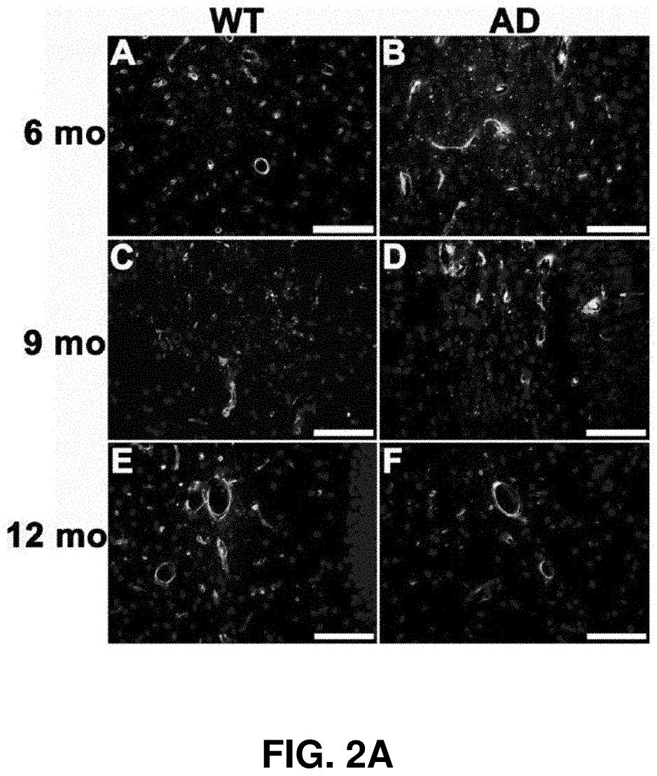

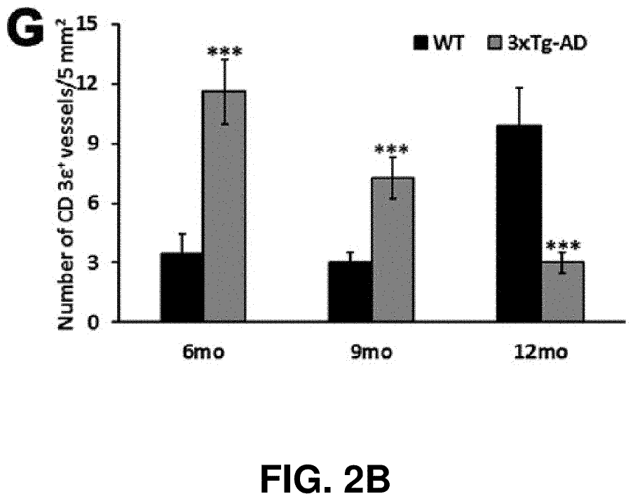

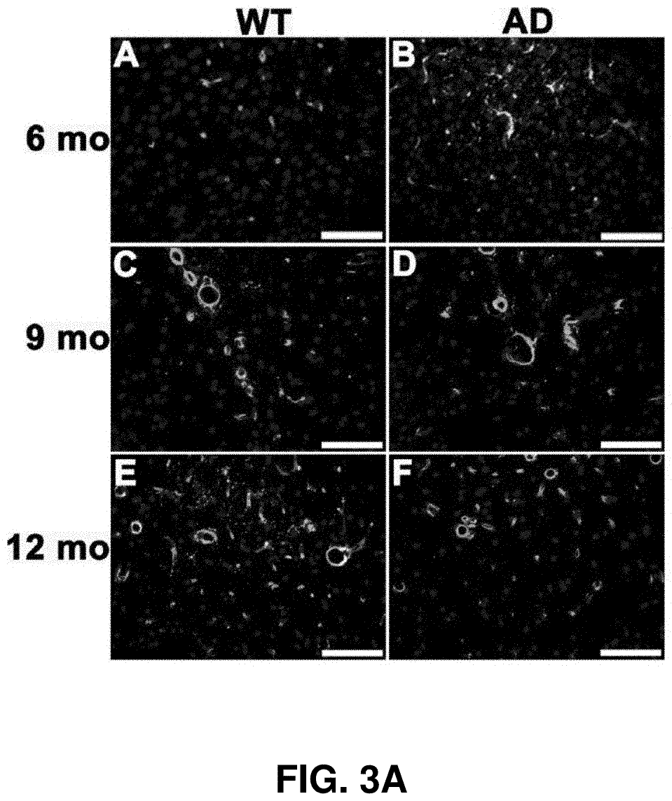

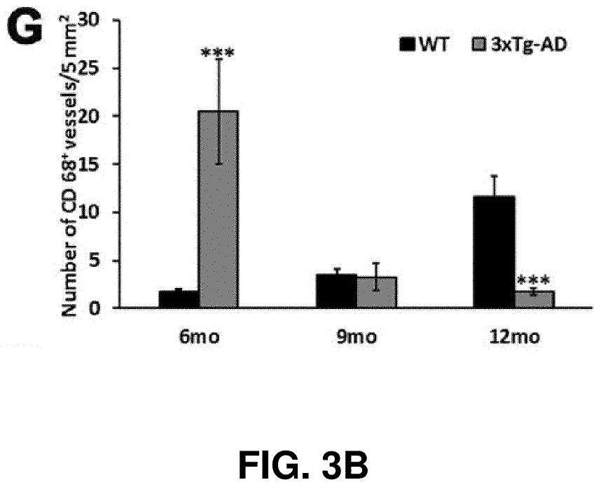

To determine the relationship of the removal of Aβ oligomers through the perivascular route and the neurovascular malfunction in AD brains, activated endothelium including activities of immune cells was detected by immunofluorescence staining. At 6 mo, robust CD3ε- and CD68-positive vessels were visualized (P<0.001) at Lectin-labeled arterioles/venules in the cortex and hippocampus of AD brains (box B of FIG. 2A and box B of FIG. 3A) in comparison with the WT brains (box A of FIG. 2A and box A of FIG. 3A). CD3ε and CD68 are usually identified as markers for immunophenotyping of cells and appeared on T cells, macrophages, monocytes, neutrophils, basophils, and large lymphocytes. Particularly at 9 months, higher CD3ε-positive vessels were still aligned with microvessels (P<0.001) in the AD brain (box D of FIG. 2A), while CD68-stained vessels disappeared in 3×Tg samples (box D of FIG. 3A). CD3ε- and CD68-stained vessels were visible at the age of 12 months in WT brains (box E of FIG. 2A and box E of FIG. 3) although they were absent in AD brains (P<0.001, box F of FIG. 2A and box F of FIG. 3). A high concentration of immune cells found in proximity to the cerebral vasculature (as shown in box B of FIG. 2A and box B of FIG. 3) suggests that endothelial activation and inflammation may be implicated in the progression of Aβ clearance and aggregation in the early AD mice.

### Different Expression Patterns of AD at Different AD Stages in 3×Tg Mice.

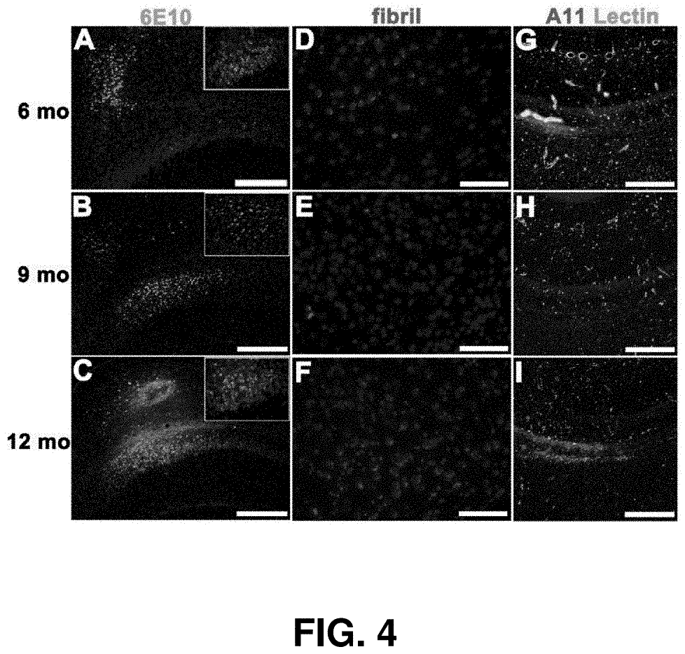

From 6 through 12 months, our data showed a progressive increase in intracellular Aβ accumulation of the cerebral cortex and hippocampus region (boxes A-C of FIG. 4) stained with 6E10 antibody. Interestingly, we found the levels of toxic Aβ oligomers transiently decreased at 9 months using A11 antibody (box H of FIG. 4) and anti-Aβ fibril antibody (that can identify all forms of Aβ, box E of FIG. 4) for immunofluorescent staining. To determine whether the transient decrease in Aβ oligomers at 9 months is associated with perivascular elimination of Aβ oligomers and the temporal profile of vascular activation, we evaluated the morphological and molecular dysfunction of cerebral vasculature in this AD mice by measuring pericyte coverage and vessel-specific miRNAs levels.

### Pericyte Coverage in 3×Tg Mice.

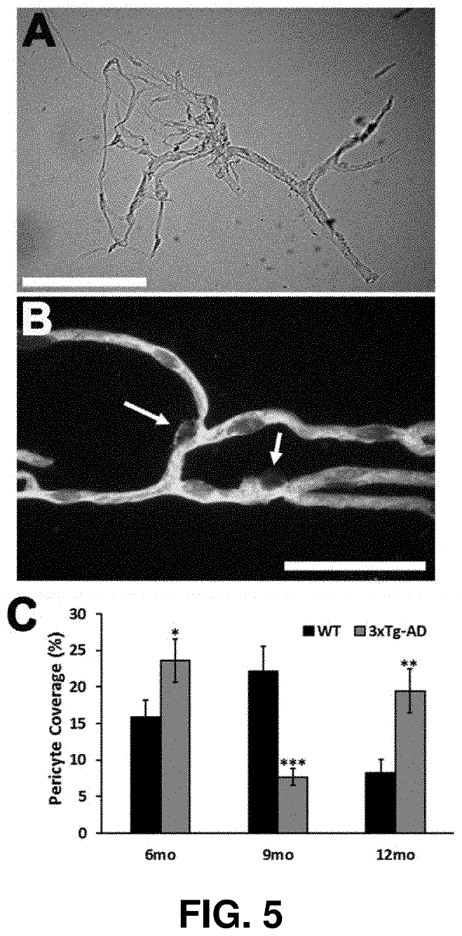

We measured the pericyte coverage in capillaries (purification >90%, box A of FIG. 5) isolated from the cerebral cortex and hippocampus of 6-12 months 3×Tg and age-matched WT mice brain, verified by anti-PDGFR-β antibody and lectin (as shown in box B of FIG. 5). The percentage of pericyte coverage was quantified as in subsection C of FIG. 5. Coincidentally, the pericyte coverage significantly increased at the age of 6 months (P<0.05), decreased at 9 months (P<0.001) and increased again at 12 months (P<0.01) in 3×Tg mice as compared to that of WT mice. Notably, an apparent increase in the pericyte coverage was observed in WT brains aged 9 mo. In addition, our findings indicate the elevation of pericyte coverage in 6 months AD brain coincident with the appearance of Aβ-loaded blood vessels and vascular activation, closely correlating with the temporal profile of Aβ, especially the decrease in Aβ oligomers of 9 months AD brains. In other words, pericyte coverage increased when intracellular Aβ appeared at 6 months as well as the activated endothelium facilitate to drive Aβ oligomer clearance from the perivascular space, resulting in the transient reduction of Aβ oligomers at 9 mo.

Levels of miRNAs in Isolated Capillaries Correlate with the Clearance of Aβ Via the Perivascular Route in 3×Tg Mice.

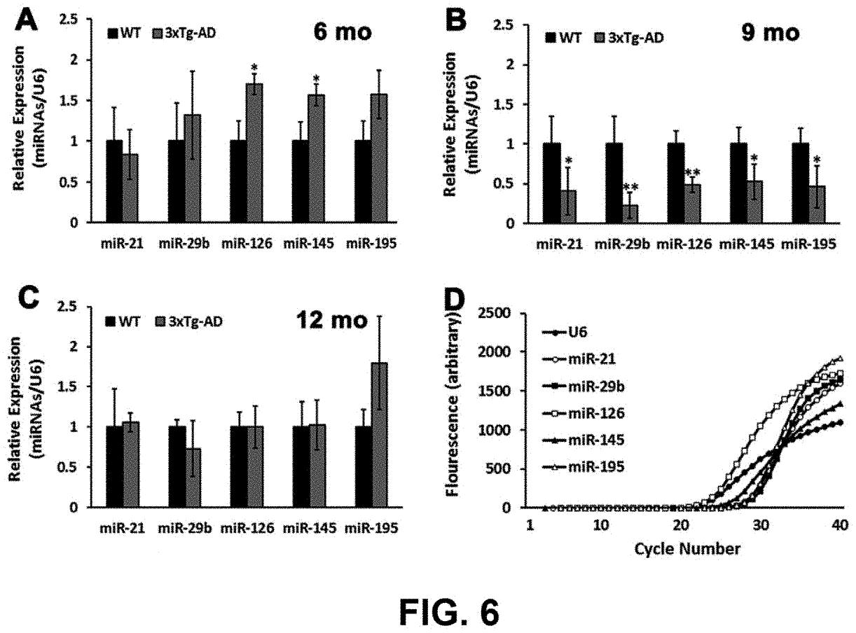

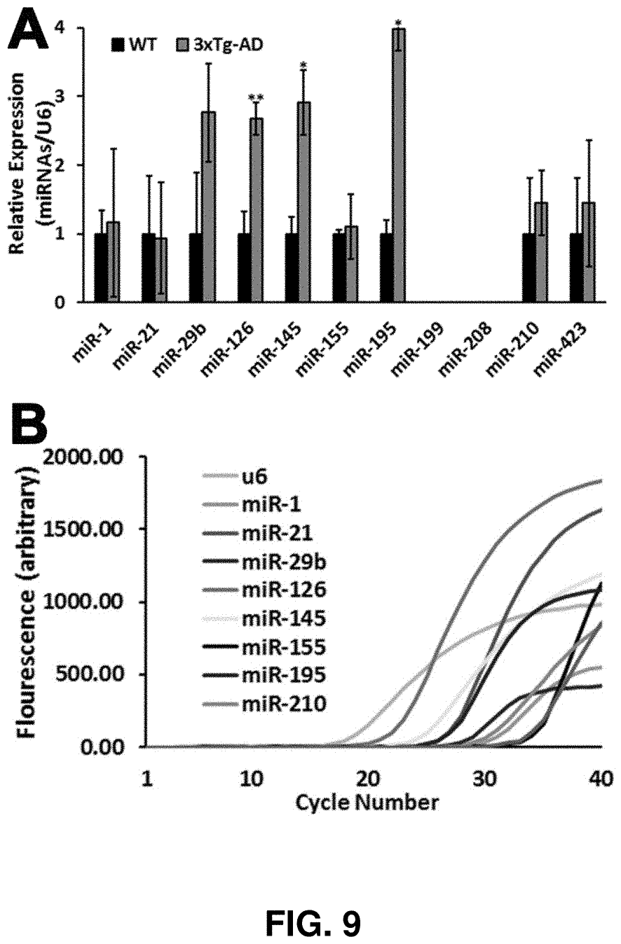

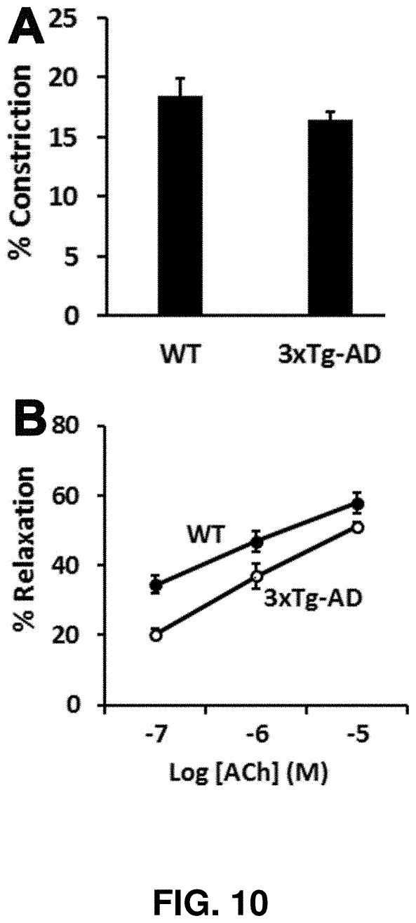

Capillaries from cerebral cortex and hippocampus were isolated using density-gradient centrifugation. A total of 11 miRNAs implicated in cardiovascular diseases and AD were screened (subsection A of FIG. 9). Based on miRNAs qPCR assay (subsection B of FIG. 9), levels of the 5 most abundant miRNAs in isolated microvessels, namely miR-126-3p, miR-145-5p, miR-195-5p, miR-21-5p and miR-29b-3p, were analyzed. These miRNAs increased (particularly miR-126 and 145, P<0.05) at the age of 6 months in AD brains (subsection A of FIG. 6). All 5 miRNAs (miR-21, 145 and 195, P<0.05; miR-29b and 126, P<0.01) significantly decreased with the reduction of Aβ oligomers (9 months, subsection B of FIG. 6), followed by a slight increase (not significantly) when Aβ fibrils appeared (12 months, subsection C of FIG. 6). As seen in subsection D of FIG. 6, miR-126 and miR-145 showed much lower threshold cycle numbers amongst the 5 selected miRNAs, which means greater abundance in isolated capillaries. This is consistent with the facts that miR-126 and miR-145 are mainly expressed in endothelial cells (ECs) and pericytes, respectively. Notably, the levels of miR-126 and 145 were significantly and consistently changed among the selected vessel miRNAs in our study. Our functional studies on middle cerebral arteries (MCAs) from 3×Tg-AD and WT mice aged 20 months using pressure myography techniques indicate there are no obvious changes on vessel SMCs function that was assessed by MCA contractions to 60 mM KCl and 10 μM phenylephrine. EC-dependent relaxations to acetylcholine were reduced in MCA from 3×Tg-AD mice. (FIG. 10). Hence, we speculate that the function of ECs was mostly impacted by Aβ deposit in 3×Tg mice. Combined with our inhibitor results (data not shown), we propose the levels of miRNAs, particularly miR-126, in isolated capillaries is strongly correlated with the clearance of Aβ from the perivascular space in 3×Tg mice.

Ghrelin Elevated miRNAs Promoted Aβ Oligomers Clearance at 9 Months in the AD Brain.

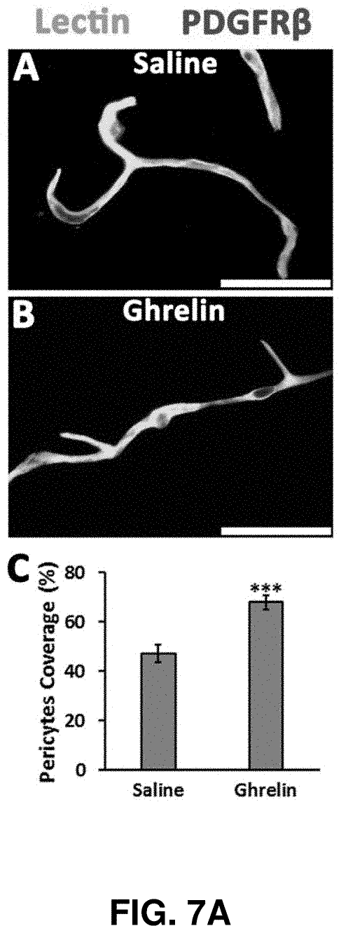

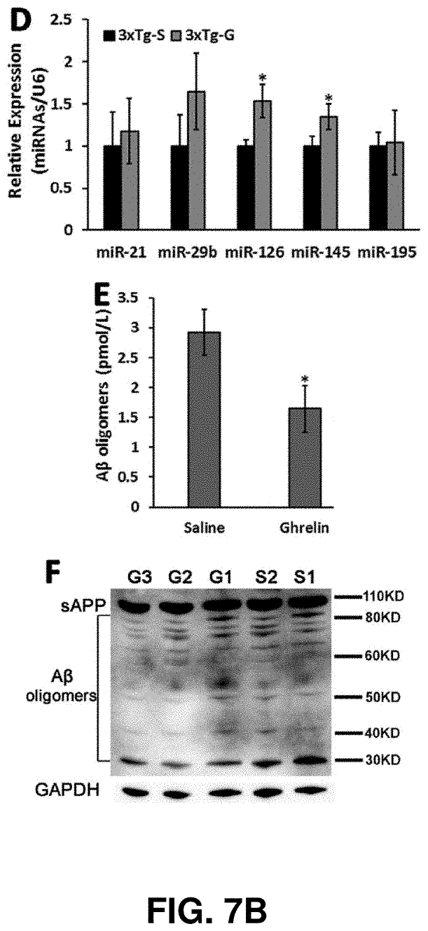

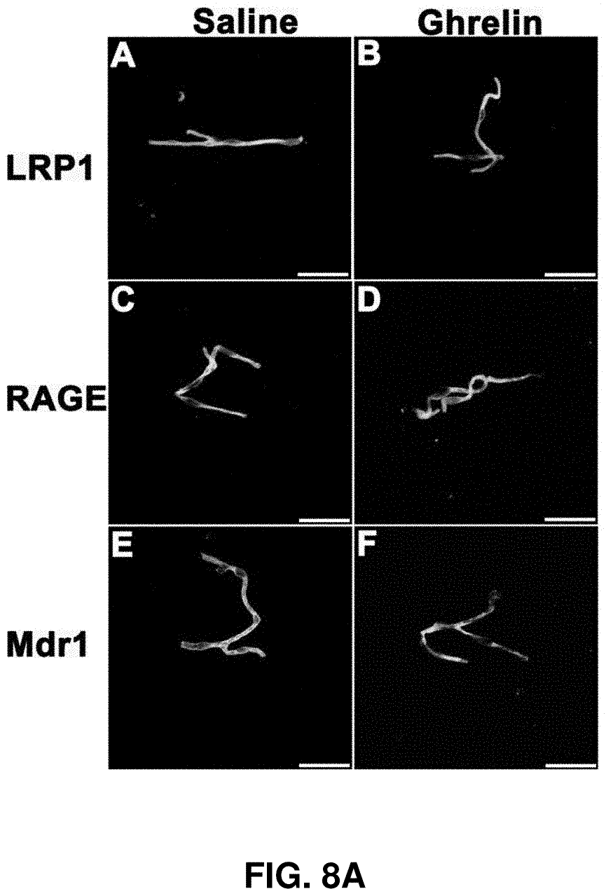

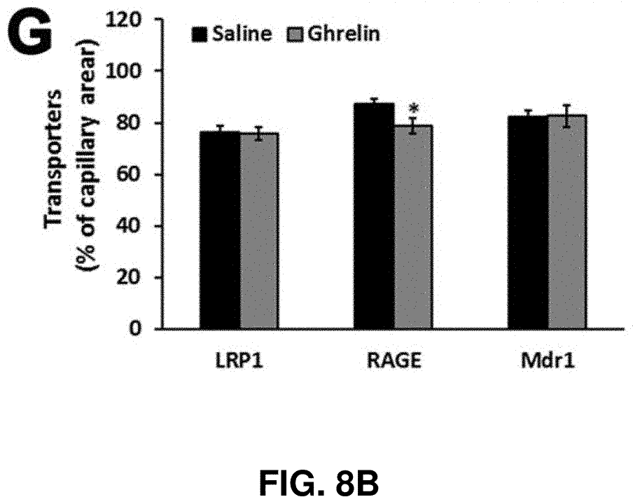

As compared with the vehicle administration, the percentage of pericyte coverage increased significantly (P<0.001) in AD mice treated with ghrelin (boxes A and B and subsection C of FIG. 7A). A significant increase in expression levels of 2 capillary miRNAs (miR126 and 145, P<0.05) from the hippocampus and cerebral cortex were observed with ghrelin treatment as compared to saline-treated AD mice (subsection D of FIG. 7B). In contrast, compared to vehicle-treated AD brains, a significantly lower level of Aβ oligomers were seen in ghrelin-treated AD brains (P<0.05) (subsections E and F of FIG. 7B). Meanwhile, the expressional levels of transporters that mediate Aβ to transport across the BBB, namely LRP1, RAGE, and Mrd1, were detected by measuring the percentages of their relative expression in the isolated capillaries. Our data reveal that the relative expression of RAGE significantly declined ˜10% in AD mice with ghrelin treatment compared to the saline-injected group (boxes D of FIG. 8A and subsection G of FIG. 8B), meaning the influx of Aβ decreased after ghrelin administration may cause the lower levels of Aβ oligomers observed in the ghrelin group.

### C. Discussion

We observed increased cerebrovascular accumulation of Aβ oligomers accompanied by the increase in activated immune cells aligned in the cerebral vasculature, elevated pericyte coverage, and up-regulation of vascular miRNAs (in particular, endothelium-specific miR-126 and 145) at 6 months in 3×Tg mice. These observations suggest that Aβ aggregation may stimulate the functional vasculature to drive Aβ oligomer elimination through the perivascular route in the early phase of AD mice. This leads to the transient decrease in Aβ oligomers and the restoration of the inflamed endothelium back to the quiescent phenotype in the next middle phase (9 months), such as the diminishing of immune cell-positive vessels, the decline of pericyte coverage, and altered capillary miRNAs expression. When Aβ fibril starts to accumulate, the pericyte coverage and capillary miRNAs levels increase again to some extent, consistent with the fact that vascular activities can continuously contribute to the pathology of AD. Ghrelin-induced miRNAs expression triggered remarkably higher pericyte coverage and reduced Aβ oligomers during the period of lowering endothelium activities in the AD brain (9 months), further supporting that the selected miRNAs are involved in regulating neurovascular activation and perivascular clearance of Aβ oligomers in AD pathogenesis. It is further implicated that the modulation of vascular miRNAs on activated vasculature and inflamed endothelium may play an important role in the pathogenesis of early AD.

ISF perivascular drainage exists as a route of metabolic byproduct removal from the brain parenchyma through perivascular spaces. Following the injection, tracers flow out of the parenchyma an hour later in the basement membranes of capillaries and in the extracellular matrix between the smooth muscle layer of the tunica media of arteries, but not along perivenous spaces. Damage to the brain microvasculature may affect Aβ perivascular elimination, promoting cerebrovascular Aβ aggregates, in turn inducing loss of vascular function and impairing angiogenesis. Aβ is present in small amounts of the normal brain and in cerebral arteries of young human individuals. Failure of Aβ clearance appears to be a major factor in the pathogenesis of the more common late-onset sporadic AD (>95% AD cases). Therapeutic strategies that facilitate the elimination of Aβ along the walls of blood vessels will expedite the discovery of novel drug targets.

Results gained from these 3×Tg mice revealed the high-level of vascular adhesion molecules and various inflammatory factors were found in hippocampus and cortex at 6 months of age. In the early phase of the 3×Tg-AD mouse model, deposits of Aβ may stimulate the inflamed BBB as a self-defense system to prevent further impairment in CNS. Combined with our findings on young and middle-aged animals, we speculate that the synthetic/activated phenotype of brain blood vessels is a form of “self-protection” against Aβ deposition in the brain, resulting in the partial clearance/degradation of Aβ by macrophages or immune cells. The protective role of extravagated macrophages, monocytes and T cells into the brain parenchyma has been demonstrated to facilitate the elimination of Aβ during the early phase of AD. The Aβ-initiated inflammation and function modification of cerebral vasculature exacerbate the disease process culminating in neuronal injury at late AD. Therefore, various investigators have proposed that blocking the migration of immune system cells into cerebral vasculature and the modification of BBB via inhibiting vascular activation may inhibit the progression of neurodegeneration and cognitive decline as a result of the neuronal toxicity of inflammatory factors, proteases and other noxious mediators released by activated brain endothelium. Nonetheless, the boost of immune activities surrounding blood vessels and promotion of the vascular activation in early/middle AD will be beneficial for both inhibiting of Aβ deposition and postpone the occurrence of AD manifestations.

This 3×Tg mouse model is a more complete model of AD that develop both Aβ and tau pathogenesis than most previous mouse strains. Our Aβ temporal profile confirmed the findings of the Laferla group (the first lab to develop 3×Tg-AD mice strain) that Aβ deposit accumulated in an age-dependent manner and Aβ oligomer dramatically reduced at 9 months. It has been proposed that Aβ fibrillization may account for the reduction of oligomers. Our observations of robust Aβ fibril immunofluorescent signals in the hippocampus at 12 months in 3×Tg mice (box F of FIG. 4) seem to support the increase in Aβ fibrillization. However, the levels of Aβ in the central nervous system (CNS) not only depend on production and fibrillization, but also on neurovascular clearance and degradation by diverse proteases in the brain parenchyma and blood. Higher immune activities from 3×Tg mice aged 6 months reported in previous studies and the transiently declined Aβ oligomers after 6 months in these triple-transgenic mice, raised our curiosity and prompted us to verify the hypothesis that vascular activation and vessel miRNAs may be involved in the metabolism mechanism of Aβ oligomer clearance through the cerebral vasculature. Our findings suggest the dynamic deposits and drainage of Aβ is directly associated with early activation of cerebral vasculature and the endothelium functional regulation. The clearance of Aβ through the cerebral vasculature and BBB functional regulation at the early stage may be mediated by the intracellular gene regulation in the vascular wall, which could be modulated by capillary miRNAs in the younger 3×Tg-AD mice brains.

Pericytes are cells uniquely located in neurovascular unit between ECs of BBB. These cells play a critical role in the modulation of the neurovascular homeostasis, including maintenance of brain microvascular stability, blood flow regulation, and clearance of toxic molecules. The integrity of BBB is maintained by the interaction between ECs and pericytes. ECs secrete PDGF-β to recruit pericytes to survive and, in turn, pericytes regulate ECs by releasing signaling molecules to maintain tight junctions. The detachment of pericytes from ECs and loss from capillaries in both hippocampus and cortex causes neurovascular degeneration. Our data showed pericyte coverage varied in line with the temporal profile of Aβ in 3×Tg mice. Aβ deposit in the perivascular space at 6 months might trigger up-regulation of vascular miRNAs, which activate the endothelium and recruit more pericytes to attach to ECs, accelerating the elimination of Aβ oligomers from the brain parenchyma and eventually lowering Aβ accumulation at 9 months.

miRNAs can contribute to regulation of the BBB function and orchestrate the various endothelium responses at the post-transcriptional levels in normal and disease brains. This work, for the first time, demonstrated the epigenetic modulation of vascular miRNAs on cerebrovascular dysfunction in AD progression and contributes to the understanding of AD pathology. As a powerful agent to regulate multiple molecular cascades and complex multi-factorial diseases, miRNAs have emerged as a class of promising targets for therapeutic intervention. Higher concentrations of oligonucleotide chemicals of miRNA modulators have been shown to affect ECs and capillaries surrounding cells in numerous cardiovascular studies. miRNA modulators in the blood can reach their targets, namely ECs and pericytes, much more easily than other potential therapies targeting neuronal or glial cells.

The modulation of 5 selected miRNAs on vascular remodeling and BBB integrity have been revealed previously. For example, miR-195 was significantly downregulated in rat brains after MCA occlusion and in hypoxia-induced human umbilical vein endothelial cells. miR-195 can inhibit human EPCs (endothelial progenitor cells) proliferation, migration, and angiogenesis under hypoxia via targeting VEGFA, while miR-145 promoted EPCs proliferation and migration in mice with cerebral infarction through the JNK signaling pathway. miR-21, as both anti- and pro-angiogenic regulator, has been shown to significantly increase after ischemic stroke, exerting opposite effects on angiogenesis in normoxia and hypoxia. In contrast, miR-21 inhibited apoptosis and promoted angiogenesis by blocking the expression of its target PTEN, and activating Akt signaling during brain injury. In ischemic stroke, overexpression of miR-29b rescued BBB disruption by downregulating aquaporin 4. Meanwhile, miR-29b improved BBB integrity by increasing the levels of matrix metallopeptidase 9 via suppressing DNA (cytosine-5)-methyltransferase 3β. miR-126 is implicated in several important aspects of vascular biology, such as angiogenesis, capillary formation, and vascular inflammation. It has been demonstrated that miR-126 is a negative regulator for MAPK and PI3K pathways via repression of SPRED1 and PIK3R2 to maintain vascular integrity and promote angiogenesis.

Over the past 10 years, ghrelin, discovered as a gastric hormone, has shown wider physiological roles in ischemia, traumatic brain injury, spinal cord injury, amyotrophic lateral sclerosis, epilepsy, Parkinson's disease, and AD. Ghrelin has been shown to exert neuroprotective effects on the AD brain and ameliorate declined cognition. Previously, no significant diminishment in Aβ plaque burden was observed in 5×FAD mice with ghrelin treatment. In contrast, our data indicated ghrelin not only up-regulated vessel miRNAs, but also attenuated Aβ oligomer load in 3×Tg mice aged 9 mo. LRP1 and Mdr1 are implicated in the effective efflux of Aβ from the brain parenchyma back to the periphery across the BBB. RAGE, involved in amyloidosis, mediated the luminal to abluminal influx of Aβ at the BBB. Tuan et al demonstrated the BBB influx/efflux of Aβ was regulated in an age-dependent fashion in 3×Tg mice. The equilibration of Aβ peptides across BBB was not disrupted until the late stages of AD (18 months). In our animals aged 9 months, ghrelin treatment disrupted the balance of influx/efflux of Aβ and attenuated the accumulation of these toxic Aβ oligomers in the parenchyma, which may be partially caused by reduced expression of RAGE. Whether RAGE expression was directly or indirectly affected by ghrelin through vessel-specific miRNAs, however, still requires further investigation.

Our study highlights the important role of the regulation of vascular miRNAs in Aβ drainage from the parenchyma and provides new knowledge to better understand AD etiology. The expression of vessel miRNAs orchestrates accurate tuning of gene expression that contributes to cerebrovascular function and BBB integrity, further influencing the extracellular Aβ clearance. The development of novel pharmacologic intervention aimed at altering the levels of cerebrovascular miRNAs will impact AD with heterogenic or epigenetic origin. Direct transport of blood vessel-specific miRNA modulators can be potentially used to control Aβ levels in the brain to treat AD manifestations and can be considered as an important and promising therapeutic approach. Therefore, the success of manipulation of vascular miRNA in AD mouse brains will stimulate the drug target discovery and facilitate the development of an effective and safe pharmaceutical intervention for clinical studies of this devastating disease.

### D. Conclusions

In summary, selected vessel-specific miRNAs, particularly miR-126 and 145, were highly correlated with the soluble Aβ clearance of brain vessels and involved in the regulation of Aβ concentration in the brains of 3×Tg-AD mice. This implicates the underlying modulation mechanism of cerebrovascular miRNAs in perivascular drainage of Aβ and brain endothelial activation.

### II. Second Study

### A. Approach

### 1. Route of Administration.

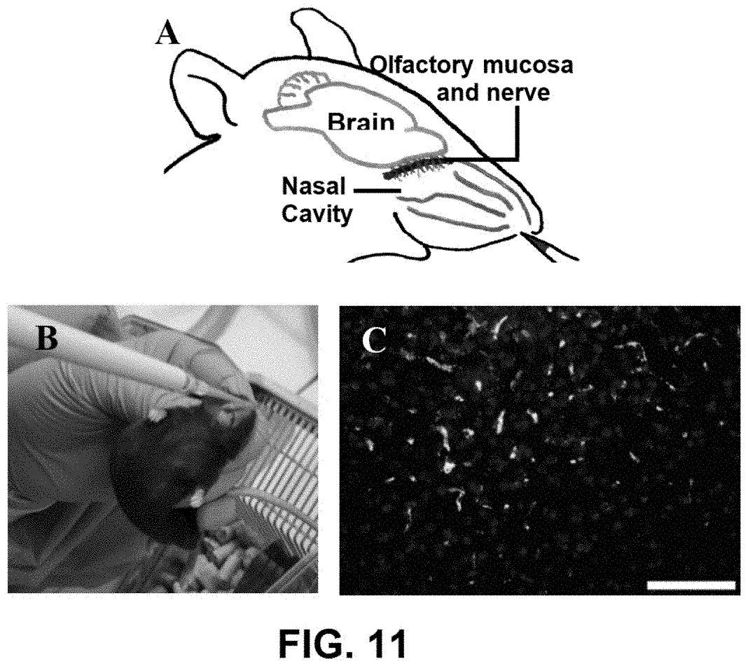

The anatomy of the nasal cavity allows the medications to pass into the brain from structures deep in the nose innervated by cranial nerves (subsection A of FIG. 11). More than 98% of all new candidate drugs for neurological disorders do not cross the BBB which limits the rate of CNS drug development. Nasal drug delivery to the brain bypasses this big challenge of BBB-mediated restriction because the traditional BBB is not present at the interface between nasal epithelium. The drug is passed through olfactory epithelium via paracellular mechanism into perineural space and transferred directly to the brain. The nose has a large surface area available for drug absorption due to the coverage of the epithelial surface by numerous microvilli and highly vascularized subepithelial layer. The intranasal route offers lower doses of drug to produce therapeutic response and quicker onsets of pharmacological activity and does not alter the normal physiological function of brain. Circumvention of the blood circulation allows reduction of the systemic exposure and hepatic/renal clearance, leading to fewer systemic side effects.

2. Preliminary Studies 2.1. High Levels of miRNAs in Isolated Capillaries Associated with the Increase in Aβ Oligomer Deposited Capillaries in Young 3×Tg Mice.

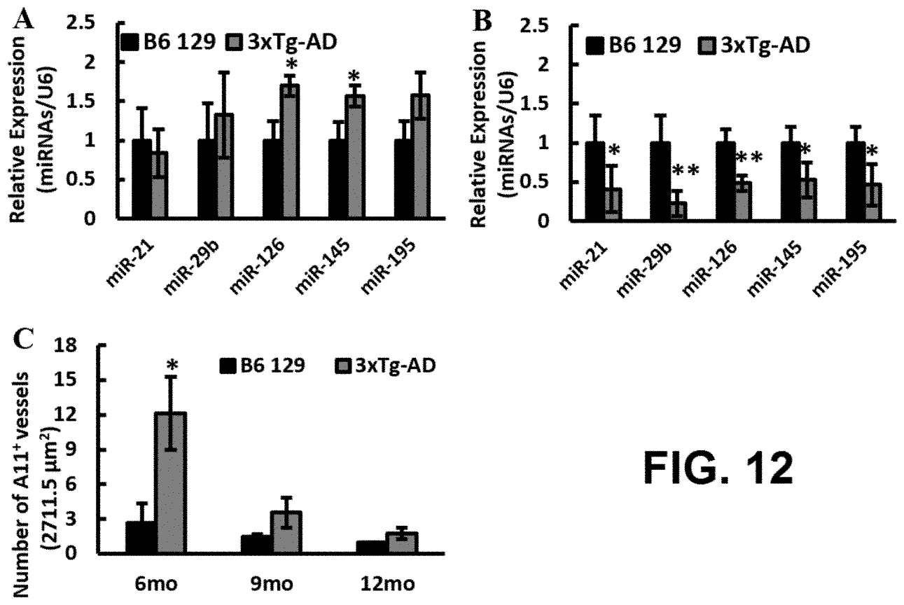

Capillaries (diameters <10 μm) from the cerebral cortex and hippocampus were isolated using density-gradient centrifugation. A total of 11 capillaries miRNAs implicated in vascular disease and AD were screened. Based on miRNAs qPCR assay, the significantly changed and most abundant 5 miRNAs with the lower threshold cycle numbers in isolated microvessels, miR-126 (3p), miR-145 (5p), miR-195, miR-21 and miR-29b (3p), were analyzed in 3×Tg-AD and B6 129 (wild-type, WT) mice. As seen in FIG. 12, miR-126 and 145 significantly increased (p<0.05) in 3×Tg mice aged 6-month old (6mo) (subsection A of FIG. 12), while all 5 miRNAs (miR-21, 145 and 195, p<0.05; miR-29b and 126, p<0.01) significantly decreased at 9 months of AD mice (subsection B of FIG. 12). Interestingly, the number of capillaries labeled with the high molecular weight Aβ oligomer-specific antibody (A11) in younger AD mice (6 months) extremely higher (p<0.05) than that of WT mice at the same age. Meanwhile, the significant increase in A11⁺ capillaries was exclusively observed at the early stage of AD mice (6 months), rather than the middle (9 months) or late (12 months) phases (subsection C of FIG. 12). These findings further suggest that the dynamic Aβ deposits are associated with BBB functional regulation and the early clearance of Aβ through cerebral vasculature, as well as may be mediated by capillary miRNAs in the younger 3×Tg-AD mice brains.

2.2. Lower miRNAs Levels in Isolated Capillaries Correlate with the Higher BBB Permeability in 6 Months of GFAP-apoE4 Mice.

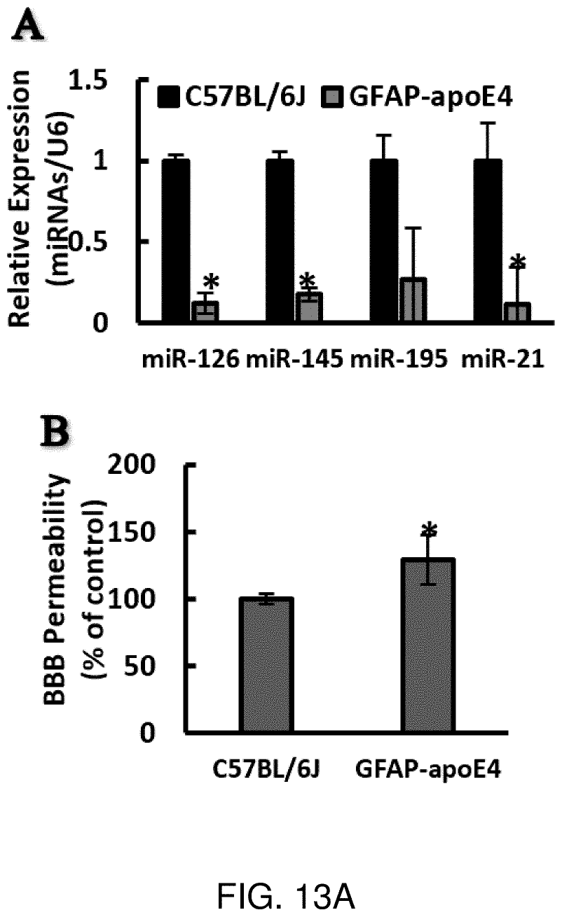

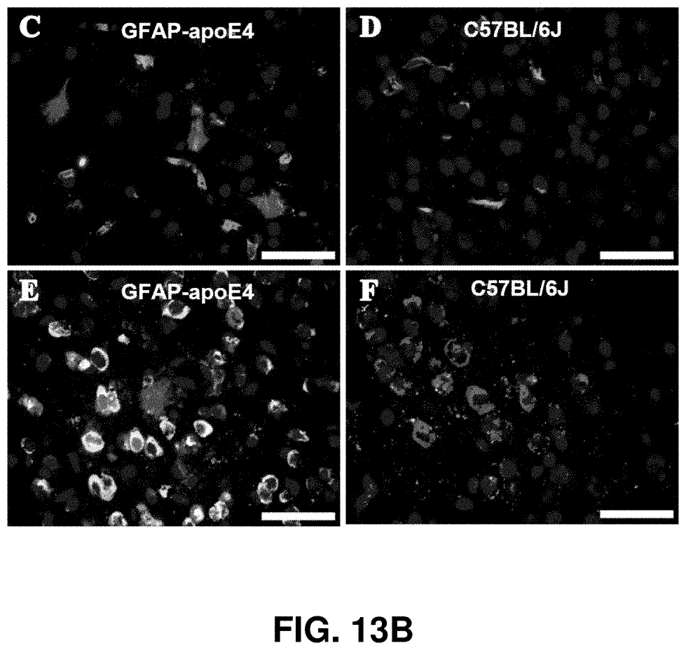

Levels of most abundant 4 microRNAs (as shown in subsection A of FIG. 13) in isolated microvessels were analyzed in the 6 months of GFAP-apoE4 (AD) and C57BL/6J (WT) mice. Our data indicate the expression of the selected miRNAs decreased where three of them downregulated significantly (miR-126 and 145, P<0.01; miR-21, P<0.05) in GFAP-apoE4 mice brains (subsection A of FIG. 13A). On the contrary, BBB permeability increased significantly (P<0.05, subsection B of FIG. 13A) in AD mice as compared with WT mice. Additionally, GFAP-apoE4 mice showed obviously extravascular thrombin accumulation (boxes C-F of FIG. 13B) co-localized with neurons. Thus, the downregulation of capillary miRNAs may be correlated with the BBB breakdown in younger apoE4 AD mice.

2.3. Effect of Ghrelin Elevated miRNAs at 9 Months and Inhibitors Blocked miR-126 and 145 at 6 Months on Aβ Oligomer Levels in 3×Tg Mice.

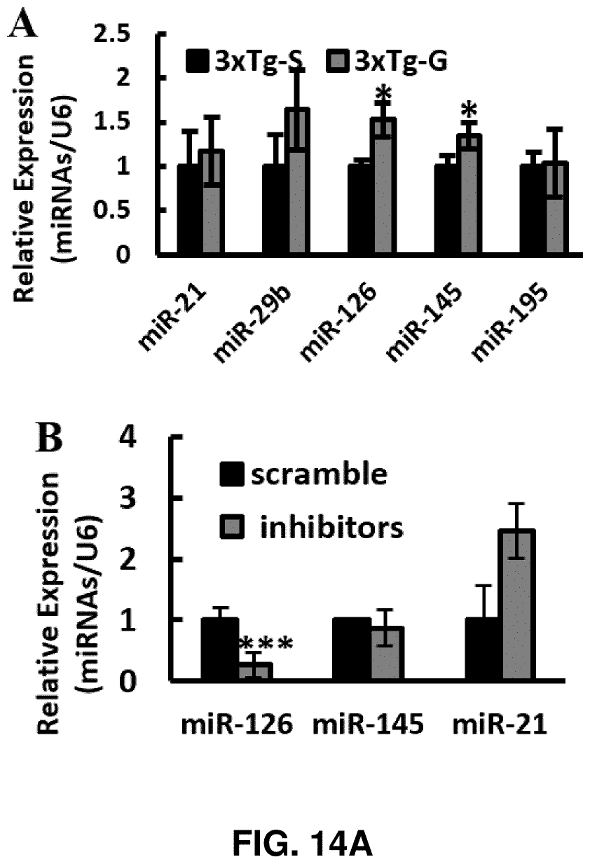

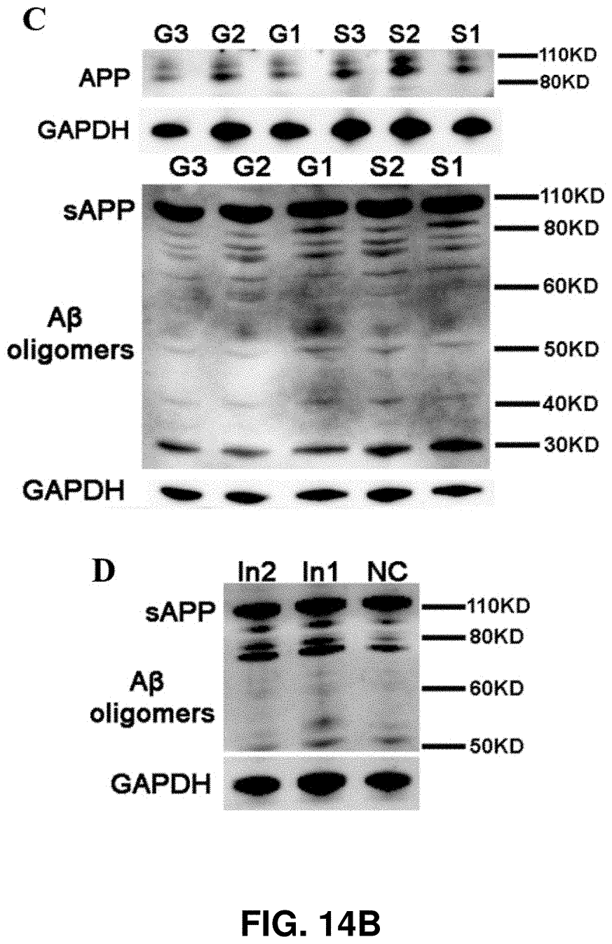

Ghrelin, a gastric hormone, has been reported to have wide physiological roles in ischemia, traumatic brain injury, Parkinson's disease and AD. It has been shown to exert neuroprotective effects on AD brain to ameliorate declined cognition, as well as promote angiogenesis and upregulate miRNAs expression in ischemic limbs. Our data indicate a significant increase in expression levels of all 5 capillary miRNAs in hippocampus and cerebral cortex of 9 months 3×Tg mice (especially for miR126 and 145, p<0.05) with ghrelin treatment (600 μg/kg per day, every 2 days for 2 weeks by S.C.) as compared to that in saline-treated AD mice (subsection A of FIG. 14A). In contrast, significantly lower Aβ levels (both APP and Aβ oligomers) were seen in ghrelin-treated AD brains (subsection B of FIG. 14A). To clarify the function of miR-126 and 145 in AD brains, 2.5 nmol of mouse miRCURY LNA™ miR-126 inhibitor combined with the same dosage of miR-145 inhibitor and 5 nmol of scramble negative control were injected into the lateral cerebral ventricle of 3×Tg-AD mice aged 6 months for 48 hrs. Our data show the levels of miR-126 was extremely reduced by more than 50% (p<0.0001) and miR-145 decreased 20% in inhibitors injected mice (subsection C of FIG. 14B). An obvious elevation in the levels of Aβ oligomers was found in inhibitor treatment groups (subsection D of FIG. 14B). These findings suggest that the higher level of miR-126 stimulated by Ghrelin was linked with reduced Aβ oligomers and the inhibition of miR-126 expression resulted in Aβ oligomers elevation.

### 2.4. the Delivery of AAV-miR-126 by the Nose-to-Brain Administration in AD Mice.

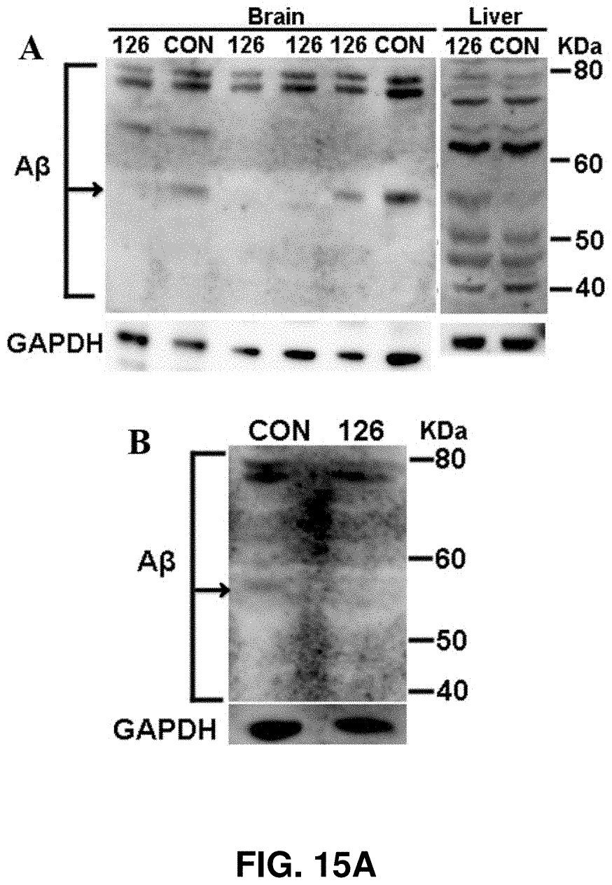

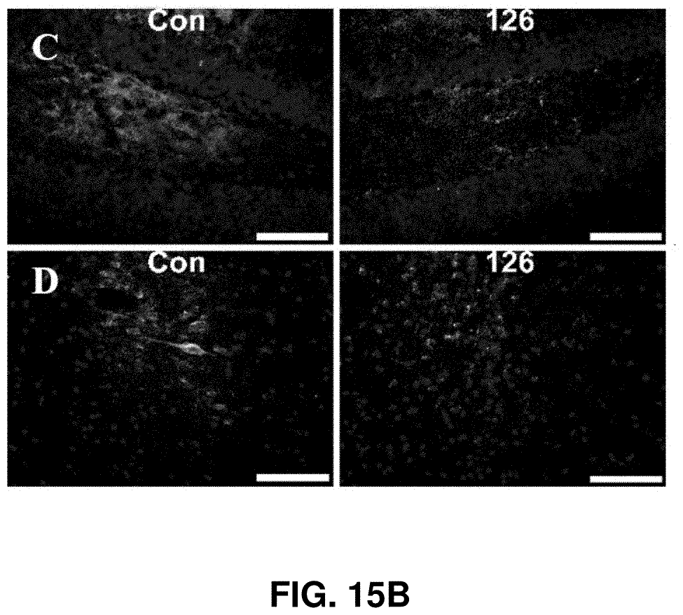

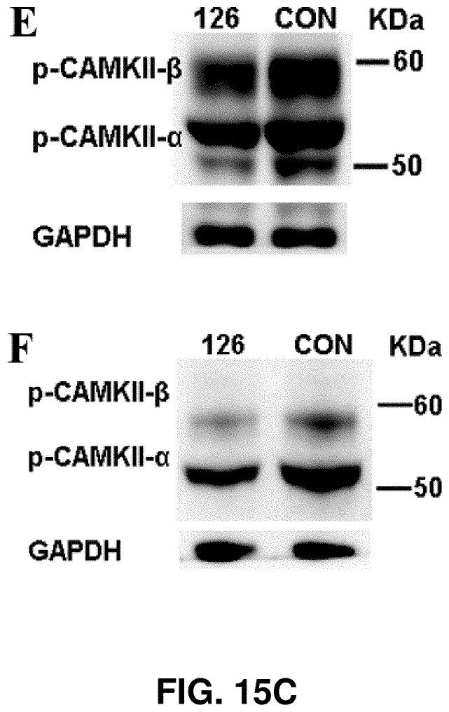

To further validate the effect of overexpressed miR-126 on the clearance of Aβ oligomers, a series of dosages and durations for rAAV1 (serotype 1, 8*10¹⁰ GC/ml) vectors containing mature miR-126 sequence (AAV1-miR-126) and blank control virus were tested on 3×Tg mice aged 6 months and apoE4 mice at 18mo. 8.5 μl of virus vectors with or without miR-126 were sprayed into each nostril of 3×Tg mice for 3 days and apoE4 mice for 9 days. After 5 days of administration, the levels of Aβ oligomers in the brain and liver were analyzed. Interestingly, the results shown in FIGS. 15A, 15B, and 15C indicate a significant elimination of the specific Aβ oligomer (between the molecular weight 50 and 60 kDa, as seen the arrow in subsections A and B of FIG. 15A) as observed in AAV-miR-126 infected 3×Tg (subsection A of FIG. 15A) and apoE4 (subsection B of FIG. 15A) AD mice brains and a significant elevation of that specific Aβ oligomer was found in miR-126 overexpressed 3×Tg mice liver (subsection A of FIG. 15A). It has been reported that the specific Aβ *56 assembly impaired synaptic functions and memory by abnormally activating NMDAR-CaMKIIα pathway and further promoted pathological tau aggregation in AD brain [31,32]. To verify the specific Aβ oligomer removed in AAV1-miR-126 infused groups is the putative Aβ *56, the signal of p-CaMKIIα were detected. The declined in activated CaMKIIα was found in the hippocampus and the cerebral cortex of miR-126 upregulated 3×Tg (boxes/rows C and D of FIG. 15B and subsection E of FIG. 15C) and apoE4 (subsection F of FIG. 15C) AD mice brains.

### 3. Research Design and Methods

### 3.1. In Vivo Validation.

MiR-126 is highly enriched in ECs and plays an important role in angiogenesis, vascular activation, and inflammation as well as maintaining vascular tone and ECs barrier function. The widely used animal model for the early-onset familial AD (FAD) is the 3×Tg-AD, a triple-transgenic mouse model of AD that harbors human mutated presenilin 1, APP and tau genes and exhibits both Aβ and tau pathological traits of FAD. Apolipoprotein ε4 (APOE4) allele is the strongest genetic risk factor for the late-onset sporadic AD (SAD) that accounts for more than 99% of AD cases. The mice model GFAP-apoE4 that carries human APOE4 allele can be used as one of the SAD mice models. Therefore, both 3×Tg-AD and GFAP-apoE4 mice strains will be utilized in this proposed study. The anesthetized mouse lies gently in the hand of the investigator, and 8.5 μl of AAV1-miR-126 (8*10¹⁰ GC/ml, purchased from Abm®, Canada) with AAV blank control will be administered dropwise into each nostril with a micropipette until the virus is completely inhaled. The enhanced fluorescent protein (eGFP) is the reporter for tracking the infection efficiency of AAV vectors. A successive 3 days administration will be conducted on 3×Tg-AD mice aged 6 months and the levels of Aβ oligomers, neuronal signaling linked to synaptic functions and cognitive behaviors as well as the safety evaluation will be examined. A series of infection for 9 consecutive days will be conducted on GFAP-apoE4 mice at the age of 18 months followed by the same experimental assessment.

### 3.2. Methods of Assessment.

On the first day after the last drop, the animals will undergo cognitive behavior evaluation by Morris water maze. A power analysis was performed to determine the number of animals required to attain statistically significant conclusions. We computed the required sample size for a student's t-test of means. The data are modeled after minimum changes of 29.2 seconds (sec) decrease of escape latency to find the platform in water maze testing caused by AAV1-miR-126 infusion. Power analysis we used the following values determined to be the number of 14 animals in each group/strain. Mean 1: 70.4 (sec); Mean 2: 41.2 (sec); Sigma: 25.1; Tail(s): 2; a: 0.05; Power: 0.85; Sample size: 14. Taking into account 10% mortality due to natural causes, we include 1 additional animal for each group per strain. Therefore, we anticipate using no more than 15 animals to obtain statistically significant data. Total 60 animals (4 groups) will be used in this proposed study, including 30 3×Tg-AD mice and 30 GFAP-apoE4 mice for AAV1-miR-126 and blank control groups, respectively.

3×Tg and apoE4 mice will be sacrificed once the behavioral studies are completed. Samples including the brain hemisphere, liver and blood will be harvested for analysis. Human Aβ oligomers of blood and extracted proteins from the brain hemisphere and liver will be measured by ELISA according to manufacturer's instructions. The changes of specific Aβ *56 for brain and liver samples from the miR-126 and blank control groups will be visualized on the membrane of western blotting using the high molecular weight Aβ oligomer-specific antibody, A11. Oligomeric Aβ *56 assembly had been described enable to bind with GluN1 directly and induce NMDAR-mediated calcium influx which selectively supra-physiologically activates Ca⁺-dependent CAMKIIα, resulting in the synapse damage and neuronal toxicity before the neuron death occurred. The relevant neuronal signaling that reflects on the synaptic function, such as the interaction of subunit of NMDAR (GluN1) with Aβ *56 and the signal of pT286-CaMKIIα, will be appraised by co-immunoprecipitation and immunoblotting, respectively. As miR-126 is involved in the vascular activation and inflammatory responses in many studies, the inflammation factors including IL-4, IL-6, IL-12A, TNF-α, CD68, CD3ε, etc. will be detected on the brain sections for each group via immunofluorescence staining using specific antibodies although none of the signals for some inflammatory factors can be detected in our preliminary data. The evaluation of the inflammatory factors mentioned and liver morphology for testing the liver toxicity can be used to estimate the drug safety for this proposed novel treatment. The difference between means will be determined using Student's t-test for two data sets. A value of P<0.05 will be indicated statistical significance.

### 3.3. Potential Pitfalls and Alternative Strategies.

To avoid the variability derived from different batches of the virus on administration, all animals used will be treated with the same one lot of AAV products (1 ml). Moreover, a small aliquot (8.5 μl) of the viruses for each mouse per day stored at −80° C. will be stable for at least one year. Some researchers addressed that GFAP-apoE4 mice did not produce the product or deposit Aβ, and we did visualize Aβ oligomers and even Aβ *56 in the brain of this strain (subsection B of FIG. 15A). “Two-hit vascular hypothesis for AD” suggests that dysfunctional cerebral vasculature could make Aβ clearance faulty and accelerate the appearance and accumulation of Aβ in the brain. Therefore, Aβ oligomers can be detected in the aging apoE4 mice (18 months). Since our aim also focused on the early prevention/therapy for AD, the age of GFAP-apoE4 strain used could be 12 months or 9 months if Aβ can be found at these ages. Thus, a lower dosage (such as the consecutive dropwise for 6 days) for younger apoE4 mice could be administered. Additionally, the synaptic proteins, such as PSD-95, Shank1, and GKAP, could be tested to evaluate the synaptic function. Dr. Chang's group has significant experiences with behavior studies. They will help to design behavior experiments for this early drug discovery study so that it can provide more valuable data as well as the formulation development.

While various embodiments of treatments of Alzheimer's disease with mRNA and gherlin and methods of performing the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A product for treating Alzheimer's disease, comprising:
recombinant adeno-associated virus (rAAV) vectors containing at least one microRNA (miRNA) sequence;
wherein the at least one miRNA sequence is selected from the group consisting of of miR-126, miR-145, miR-195, miR-21, and miR-29b.

2. The product of claim 1, wherein the at least one miRNA sequence is miR-126.

3. The product of claim 2, wherein the at least one miRNA sequence further comprises at least one additional miRNA sequence.

4. The product claim 1, configured for intranasal administration

5. The product of claim 1, wherein the at least one miRNA sequence is miR-145.

6. The product of claim 5, wherein the at least one miRNA sequence further comprises at least one additional miRNA sequence.

7. The product of claim 1, wherein the miRNA sequence is vessel-specific.

8. A method, comprising the step of:
Administering the product of claim 1 to an individual having Alzheimer's disease to treat the Alzheimer's disease.

9. The method of claim 8, wherein the step of administering is performed using intranasal administration.

10. A method of treating Alzheimer's disease, comprising the step of:
administering at least one recombinant adeno-associated virus (rAAV) vector to an individual having Alzheimer's disease, wherein the rAAV vector comprises at least one microRNA (miRNA) sequence;
wherein the at least one miRNA sequence is selected from the group consisting of of miR-126, miR-145, miR-195, miR-21, and miR-29b.

11. The method of claim 10, wherein the step of administering is performed using intranasal administration.

12. The method of claim 10, further comprising the step of:
administering ghrelin to the individual prior to the step of administering at least one rAAV vector.

13. The method of claim 10, further comprising the step of:
administering ghrelin to the individual after the step of administering the at least one rAAV vector.

14. The method of claim 10, wherein the step of administering further comprises administering ghrelin.

15. The method of claim 10, further comprising the step of administering ghrelin subcutaneously.
